(19) United States
(12) Patent Application Publication
(10) Pub. No.: US 2022/0104777 A1
(43) Pub. Date: Apr. 7, 2022

(54) A BREATH INDICATOR

(71) Applicant: Fisher & Paykel Healthcare Limited, Auckland (NZ)

(72) Inventors: Jonathan Mark CHURCH, Auckland (NZ); Zak Jake FLINTOFF, Auckland (NZ); Brenda Louise BAIN, Auckland (NZ); Michelle Lesley MUIR, Auckland (NZ); Daniel Charles WILSON, Auckland (NZ)

(21) Appl. No.: 17/309,386

(22) PCT Filed: Nov. 15, 2019
(86) PCT No.: PCT/IB2019/059813

§ 371 Date: May 24, 2021

Related U.S. Application Data
(60) Provisional application No. 62/772,014, filed on Nov. 27, 2018.

(51) Int. Cl. A61B 5/00; A61B 5/097; A61B 5/08

(52) U.S. Cl. CPC A61B 5/742; A61B 5/082; A61B 5/097

(57) ABSTRACT

This invention relates to a breath indicator for providing an indication of an inhalation and/or exhalation state of a user. Said breath indicator comprising a body comprising a gas sampling portion configured for receipt of a user's inhalation and/or exhalation gases, and an indicator portion. Said gas sampling portion in fluid communication with said indicator portion. Said indicator portion comprising at least a first region and at least a second region, the first region comprising a gas parameter detecting material capable of changing between an initial visual state and a subsequent visual state indicative of detection of a gas parameter of inhalation and/or exhalation gases of said user; and wherein said initial visual state and/or said subsequent visual state of the first region visually contrasts with a visual state of at least said second region.

### TECHNICAL FIELD

The present disclosure generally relates to a breath indicator. The breath indicator may be used in conjunction with or as part of breathing assistance apparatus, such as used in resuscitation of a patient, to indicate whether the patient is breathing.

### BACKGROUND ART

It is known to apply Positive End Expiratory Pressure (PEEP) and controlled Peak Inspiratory Pressure (PIP) during respiration, resuscitation or assisted respiration (ventilation) for patients who require breathing assistance. In applying PEEP, the patient's upper airway and lungs are held open by the applied pressure.

It is important for a medical professional to be able to establish and verify the breathing of the patient. When undergoing assisted ventilation or respiration, the patient will receive a continuous flow of gases to their airway. These gases will either be atmospheric, atmospheric with supplementary added oxygen, or (rarely) pure oxygen. When the patient exhales against the continuous flow of gases, they will breathe out gases that have a higher concentration of carbon dioxide than those which enter their lungs.

It is known to use carbon dioxide detectors with adult ventilation systems and as part of breathing masks. U.S. Pat. No. 4,945,918 discloses using a CO₂ detector with a ventilation system to detect the patient's circulatory status. U.S. Pat. No. 5,857,460 discloses a mask with a gas sensor positioned on the mask.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

Further aspects and advantages will become apparent from the ensuing description which is given by way of example only.

### SUMMARY

The present disclosure seeks to provide a breath indicator, either alone or to be provided as part of a breathing assistance system or apparatus, that will at least go some way towards improving on the above or which will at least provide the public or the medical profession with a useful choice.

In a first aspect, there is provided a breath indicator for providing an indication of an inhalation and/or exhalation state of a user, said breath indicator comprising:

a detecting material capable of changing colour or visual state from a first colour or an initial visual state, to a second colour or a subsequent visual state, according to inhalation and exhalation, said detecting material housed within a body, wherein said body comprises a first port configured for receipt of a user's inhalation and exhalation gases and at least a second port to provide for a venting of said gases from said body, and

a component comprising at least one indicium changeable as to a colour or a contrast state according to a change in colour or contrast state of said detecting material with said user's inhalation and exhalation gases.

In a second aspect, there is provided a breath indicator for providing an indication of an inhalation and/or exhalation state of a user, said breath indicator comprising:

a body comprising a first port configured for receipt of a user's inhalation and exhalation gases and at least a second port to vent said gases from said body,

wherein said body comprises a detecting material capable of changing from a first colour or an initial visual state, to a second colour or a subsequent visual state according to a change in gas composition or according to a gas parameter of a user's inhalation and/or exhalation gases, and

a component comprising at least one indicium configured to provide a colour or a visual contrast state change according to a change in colour or a visual contrast state of said detecting material based on said change in gas composition or according to a detected gas parameter.

The detecting material may detect a parameter of a gas. The parameter may be one or more of: a gas temperature, a gas humidity content, a gas composition.

The detecting material may be a carbon dioxide detecting material or detector.

The detecting material may be of a first colour or a first or an initial visual state when a carbon dioxide concentration is about the same or substantially similar to atmospheric air.

The detecting material may be of a first colour or a first or an initial visual state when exposed to gas containing or comprising up to about 0.03% or about 0.04% carbon dioxide (atmospheric).

The detecting material may be of a second or a different colour or a second or a subsequent visual state when carbon dioxide concentration is greater than atmospheric air.

The detecting material may be of a second colour or a second or a subsequent visual state when exposed to gas containing or comprising greater than about 0.2% or greater of a carbon dioxide concentration.

The detecting material may be of a second colour or a second or a subsequent visual state when exposed to gas containing or comprising about 1.2% or greater of a carbon dioxide concentration.

The detecting material may be of a second colour or a second or a subsequent visual state when exposed to gas containing or comprising about 2.5% or greater of a carbon dioxide concentration.

A first colour or a first or an initial visual state of said detecting material may be one of: blue or purple.

A second colour or a second or a subsequent visual state of said detecting material may be one of: yellow or orange.

A change from a first colour or a first or an initial visual state to or between a second colour or a second or a subsequent visual state of said detecting material may be based on a chemical reaction of said detecting material.

The chemical reaction may be reversible.

A change from a first colour or a first or an initial visual state to or between a second colour or a second or a subsequent visual state of said detecting material may be visually observable.

The indicia may provide for a greater contrast of colour or greater visibility or greater visual contrast state with respect to said detecting material to indicate either of a detected greater or lesser parameter of fluid.

The parameter of fluid may be of a gas, and may be one or more of a gas temperature, a gas humidity content, a gas composition.

The indicia may provide for a greater contrast of colour or greater visibility or greater visual contrast state with respect to said detecting material to indicate either of a detected greater or lesser concentration of carbon dioxide relative to atmospheric carbon dioxide concentration.

The indicia may provide for a greater contrast of colour or greater visibility or greater visual contrast state with respect to said detecting material to indicate either of a detected greater or lesser concentration of one or more detected gases forming the gas composition.

The detected gases may be one or more of: carbon dioxide, oxygen, nitrogen, argon or any other gas normally present in air or inhalation/exhalation gases.

The indicia may provide for:

a first contrast state or a first or an initial visual state with respect to said detecting material when carbon dioxide concentration is the same or less than an atmospheric concentration of carbon dioxide, and/or

a second contrast state or a second or a subsequent visual state with respect to said detecting material when carbon dioxide concentration is greater than an atmospheric concentration of carbon dioxide.

The indicia may provide for:
a first contrast state or a first or an initial visual state with respect to said detecting material when a detected gas parameter is the same or less than a detected gas parameter of atmospheric gases, and/or

a second contrast state or a second or a subsequent visual state with respect to said detecting material when a detected gas parameter is greater than a detected gas parameter of atmospheric gases.

A first and a second indicia contrast states may be degrees of visibility.

A second indicia contrast state may be of a greater visibility or observability (i.e. appears more easily visible or optically detectable) with respect to said detecting material.

A second indicia contrast state may be significantly more visible than first indicia contrast state.

A first indicia contrast state may be when said detecting material is a first colour.

A first indicia contrast state may be when said detecting material is exposed to gas containing or comprising up to about 0.03% or about 0.04% carbon dioxide (atmospheric).

A first indicia contrast state may be when carbon dioxide concentration is substantially the same as atmospheric air.

A change from a first indicia contrast state to a second indicia contrast state may be based on a chemical reaction of said detecting material.

A change from a first indicia contrast state to a second indicia contrast state may be a visually or an optically observable change in contrast (e.g. optically observable by a person).

At least one indicium may be an icon, optionally said icon resembles a pair of lungs (such as human lungs), or wind, or a heart or a graphical smiley face or other positive display of a detected carbon dioxide concentration.

The at least one indicium may be printed or mounted directly upon said component.

At least one indicium may be printed or mounted directly upon said detecting material.

The detector material may visually surround said indicia.

The detector material may visually border more than one edge or perimeter portion of said indicia.

The component may be one or more of: the detecting material, a breath indicator cap or another separate component of a breath indicator assembly.

The breath indicator may further comprise: an elongate body, a cap, a sensor or detector portion comprising said detecting material, said sensor or detector portion positioned to be exposed to a gas flow through said elongate body into a region housed by said cap.

The cap may include or comprise a radially inwardly extending projection portion for holding or retaining the cap and the elongate body together in an engaged configuration.

The projection portion may engage with an exterior portion of said elongate body.

The projection portion may extend circumferentially inwardly around the cap.

The cap may be receivable of an end of said elongate body, said cap configured to be retained upon said end of said elongate body.

The end of said elongate body may be an end configured to receive or locate said sensor or detector portion.

The end of said elongate body may be configured to provide a platform to receive or locate said sensor or detector portion.

The end of said elongate body and said cap, when in said engaged configuration may provide a housing for said sensor or detector portion.

The elongate body may comprise a hollow passageway extending between ends of said elongate body, a first end of said elongate body provided as a gas sampling end and a second end of said elongate body provided as a region for receipt or locating of said sensor as a detector portion.

The second end of said elongate body may comprise a platform region upon which said sensor or detector portion is to be received or located, said hollow passageway terminating into said platform region.

A detecting material may be received or located in a void space provided between an end of said elongate body and an internal wall surface of said cap (when said cap is in-situ or in an engaged or an assembled configuration with said elongate body).

A component with at least one indicia printed or mounted directly thereon may be received or located in a said void space between an end of said elongate body and an underside or an internal wall surface side of said cap (when said cap is in-situ or in an engaged or an assembled configuration with said elongate body).

The component may be one or more of: the sensor, the detecting material, the cap, or the component may be provided as a separate item from each of the sensor, detecting material, cap and/or component).

The cap may be formed, at least in part, of an optically transparent material.

The cap or a portion thereof, may provide an optical magnification capability of said sensor and/or said indicia located beneath said cap.

The cap may comprise a shell or lid portion.

The cap or a portion thereof may provide for an increased viewing angle of said sensor and/or said detecting material located beneath said cap.

The cap or a portion of the cap may be substantially or partly composed of a substantially optically clear material.

The cap may have at least a first portion comprising a substantially optically clear material or texture, and at least a second portion comprising opaque or translucent material or texture.

The substantially opaque or translucent second portion may surround the first portion, such that a feature securing the cap to the breath indicator is obscured.

The substantially opaque or translucent second portion may be provided by a roughened texture material of the cap.

The elongate body may comprise a sampling end. The sampling end may comprise a conduit with an open end defining a gas inlet and a flow path extending from said open end to said sensor or detector portion.

The elongate body may comprise a conduit with an open end defining a gas inlet and a flow path extending from said open end to said sensor or detector portion.

The flow path may comprise a passageway or conduit extending from said open end of the gas inlet to said sensor or detector portion.

The open end or gas inlet may be provided as a gas sampling or gas receiving end of said elongate body.

The flow path may provide for fluid (e.g. gas) communication between a gas sampling end of said elongate body and a gas sensing end (or the sensor end) or gas detector portion of said elongate body.

The elongate body may comprise a terminal end of a flow path or passageway or conduit extending from an open end or a gas inlet into said elongate body, said terminal end terminated by said cap (when said cap is in-situ or in an engaged or an assembled configuration with said elongate body).

The terminal end may comprise at least one gas flow port for discharging a gas flow directed from a gas sampling end of said elongate body through said terminal end to atmosphere.

The gas flow port may comprise a vent or outlet to atmosphere.

The one or more gas flow port(s) may be one or more apertures provided as a vent hole or vent holes.

The one or more gas flow ports may control the rate of gas flow or venting or allowed leak from said breath indicator.

The one or more gas flow ports may be sized to provide for a controlled vent or a pre-determined gas flow rate of gas.

The one or more gas flow ports may be sized to provide for a controlled vent rate or a pre-determined gas flow rate of gas.

A gas flow or vent rate may be about 0 litre/minute to about 3 litre/minute, preferably about 1 litre/minute.

A gas flow or vent rate may be within the operational parameters of PEEP and/or PIP when the breath indicator is used in combination with a patient interface.

A gas flow port may be an aperture of a diameter of about 0 mm to about 1 mm, or about 0.7 mm.

The one or more gas flow ports may be provided through a portion of the elongate body provided in fluid communication with a gas flow to be sampled or sensed, said one or more gas flow ports being downstream of a gas sampling end of said elongate body and/or downstream of said sensor.

The one or more gas flow ports may be located substantially adjacent to or about a stopping flange portion of the elongate body.

The stopping flange may be an enlarged region or portion of said elongate body.

The stopping flange may be a region or portion of said elongate body that is engageable with a portion of a device to which said indicator is attachable or engageable, to provide for a located position of said elongate body relative to a said device or to provide for a maximum or a predetermined length (or depth) of insertion of said elongate body into a device.

A length of the body may be about 20 mm to about 60 mm, or about 40 mm.

A length of height of a cap may be about 2.5 mm to about 10 mm, or about 5 mm.

The one or more gas flow ports may be substantially circular shaped aperture(s).

There may be a single (one or sole) gas flow port.

The indicator may further comprise:

a body comprising a gas sampling portion and a gas sensing portion or detector portion, said gas sampling portion provided in fluid communication with said gas sensing portion or detector portion, wherein a gas detector is housed within said gas sensing portion or detector portion, the gas detector comprising a material providing a visually detectable change in colour or visual state in response to a parameter of a gas to be detected (such as a concentration of a gas to be detected), and wherein a pre-determined indicia is provided on or adjacent to said gas detector.

The elongate body may further comprise:

a gas sampling portion and a gas sensing portion or detector portion, said gas sampling portion being provided in fluid communication with said gas sensing portion or detector portion, wherein a gas detecting material is housed within said gas sensing portion, the gas detecting material providing for a visually detectable change in colour or visual state in response to a concentration of a gas to be detected, and wherein a pre-determined indicia is provided directly upon said gas detector.

The pre-determined indicia may be of a pre-determined shape and/or colour.

The indicia may be a shape resembling an icon.

The icon may be indicative of one or more of breathing, lungs, wind, a heart, graphical smiley face or other positive display of a detected parameter of a gas, such as a carbon dioxide concentration or another visual cue.

The indicia may be directly imprinted or printed or laid upon said gas detector, or provided as a separate component located in communication with said gas detector.

The gas detecting material may be a sensor, said sensor located to be exposed to a gas flow through or about said body.

The body may be an elongate body.

The elongate body may taper from a relatively smaller outer diameter substantially at or toward said gas sampling portion to a relatively larger outer diameter substantially at or toward said gas sensing portion or detector portion.

The gas sampling portion may comprise a lead-in portion.

The lead-in portion may be a region of said elongate body being of a substantially narrowed (or more narrowed) outer diameter, or said lead-in portion is a tip or an end of said elongate body comprising of a chamfered edge or substantially radially inward curvature so as to assist with access or initial insertion of said gas sampling end into a device, such through as a duck-bill valve of a device.

A substantially tapered configuration of said body (such as an elongate body of said indicator) may extend from at or substantially toward said gas sampling portion toward said gas sensing portion, or detector portion, about one quarter, or about one third, or about one half, two-thirds, three-quarters, or a whole of, the length of said elongate body.

The cap may be provided as a housing to said sensor or detector portion. Optionally the sensor may comprise a gas detecting material, or gas detector. The indicia may be applied to the cap.

The cap may be detachable from said body.

The body may comprise a gas sampling portion and a gas sensing portion or detector portion, said gas sampling portion being provided in fluid communication with said gas sensing portion or detector portion via an elongate body, said gas sensing portion or detector portion housing a gas detector, the gas detector comprising a material providing a visually detectable change in response to a gas parameter, such as a concentration of a gas to be detected, said gas sensing portion or detector portion being distal from said gas sampling portion, and wherein said elongate body providing for said gas sampling portion distal of said gas sensing end or detector portion, and wherein said elongate member is of a substantially tapered configuration, tapered from a relatively smaller outer diameter at or substantially toward said gas sampling end to a relatively larger outer diameter as said elongate body progresses toward said gas sensing portion or detector portion.

The visually detectable change may comprise a change in colour, shade, contrast or brightness.

The gas sampling end may comprise a lead-in portion.

The lead-in portion may be a region of said elongate body being of a substantially narrowed (or more narrowed) external diameter, or said lead-in portion is a tip or end of said elongate body comprising of a chamfered edge or substantially radially inward curvature so as to assist with access or initial insertion of said gas sampling end into a device, such through as a duck-bill valve of a device.

The substantially tapered configuration may extend from at or substantially toward said gas sampling portion toward said gas sensing portion, or detector portion, about one quarter, or about one third, or about one half, two-thirds, three-quarters, or a whole of, the length of said elongate body.

A cap may be provided as a housing to said gas detector.

The cap may be removably attachable to said gas sensing portion or gas detector portion.

In a third aspect there is provided a breath indicator for providing an indication of an inhalation and/or exhalation state of a user, said breath indicator comprising: an elongate body, a cap, a sensor or detector portion comprising a detecting material, said sensor or detector portion positioned or located to be exposed to a gas flow through said elongate body into a region housed by said cap, wherein said detecting material is capable of changing colour from a first colour, or a first contrast state or a first or an initial visual state, to a second colour, or a second contrast state or a second or a subsequent visual state, according to a change in a gas parameter, such as a gas composition, associated with inhalation and exhalation, said detecting material housed within a body, wherein said body comprises a first port configured for receipt of a user's inhalation and exhalation gases and at least a second port to provide for a venting of said gases from said body, and a component comprising at least one indicium changeable as to contrast according to a change in colour, or contrast or visual state, of said detecting material with said user's inhalation and exhalation gases.

The cap may include or comprise a radially inwardly extending projection portion for holding or retaining the cap and the elongate body together in an engaged configuration.

The projection portion may engage with an exterior portion of said elongate body.

The projection portion may extend circumferentially inwardly around the cap.

The cap may be receivable of an end of said elongate body, said cap configured to be retained upon said end of said elongate body.

The end of said elongate body may be an end configured to receive or locate said sensor or detector portion.

The end of said elongate body may be configured to provide a platform to receive or locate said sensor or detector portion.

The end of said elongate body and said cap, when in said engaged configuration may provide a housing for said sensor or detector portion.

The elongate body may comprise a hollow passageway extending between ends of said elongate body, a first end of said elongate body provided as a gas sampling end and a second end of said elongate body provided as a region for receipt or locating of said sensor or detector portion.

The second end of said elongate body may comprise a platform region upon which said sensor or detector portion is to be received or located, said hollow passageway terminating into said platform region.

A detecting material may be received or located in a void space provided between an end of said elongate body and an internal wall surface of said cap.

A component with at least one indicium thereon may be received or located in a said void space between an end of said elongate body and an underside or an internal wall surface side of said cap.

The component may be one or more of: the sensor or detector portion, the detecting material, the cap, or the component may be provided as a separate item from each of the sensor, detecting material, cap or component).

The cap may be formed, at least in part, of an optically transparent material.

The cap may provide for an optical magnification capability of said sensor and/or said indicia to be located beneath said cap.

The elongate body may comprise an open end or a gas inlet and a flow path or passageway or conduit extending from said open end or the gas inlet to said sensor or detector portion.

The open end or gas inlet may be provided as a gas sampling or gas receiving end of said elongate body.

The flow path or passageway or conduit may provide for fluid (i.e. gas) communication between a gas sampling end of said elongate body and a gas sensing end (or the sensor end) or detector portion of said elongate body.

The elongate body may comprise a terminal end of a flow path or passageway or conduit extending from an open end or a gas inlet into said elongate body, said terminal end terminated by said cap.

The terminal end may comprise at least one gas flow port to atmosphere for discharging of a gas flow directed from a gas sampling end of said elongate body through said terminal end to atmosphere.

The sensor or detecting material may be located or positioned within a flow path of a gas to be sampled.

The detecting material may be capable of changing colour from a first colour to a second colour according to inhalation and exhalation, said detecting material housed within said elongate body, wherein said elongate body comprises a first port configured for receipt of a user's inhalation and exhalation gases and at least a second port to provide for a venting of said gases from said body, and a component comprising at least one indicia changeable as to contrast according to a change in colour of said detecting material with said user's inhalation and exhalation gases.

The detecting material may be capable of changing between a first colour or a first or an initial visual state and a second colour or a second or a subsequent visual state according to inhalation and exhalation, said detecting material housed within said elongate body, wherein said elongate body comprises a first port configured for receipt of a user's inhalation and exhalation gases and at least a second port to provide for a venting of said gases from said body, and a component comprising at least one indicia changeable as to contrast according to a change in colour or visual state of said detecting material with said user's inhalation and exhalation gases.

The detecting material may detect a parameter of a gas. The parameter may be one or more of: a gas temperature, a gas humidity content, a gas composition.

The detecting material may be a carbon dioxide detecting material or detector.

The detecting material may be of a first colour or a first or an initial visual state when a carbon dioxide concentration is about the same or substantially similar to atmospheric air.

The detecting material may be of a first colour or a first or an initial visual state when exposed to gas containing or comprising up to about 0.03% or about 0.04% carbon dioxide (atmospheric).

The detecting material may be of a second or a different colour or a second or a subsequent visual state when carbon dioxide concentration is greater than atmospheric air.

The detecting material may be of a second colour or a second or a subsequent visual state when exposed to gas containing or comprising about 2.5% or greater of a carbon dioxide concentration.

A first colour or a first or an initial visual state of said detecting material may be one of:

blue or purple.

A second colour or a second or a subsequent visual state of said detecting material may be one of: yellow or orange.

A change from a first colour or a first or an initial visual state to or between a second colour or a second or a subsequent visual state of said detecting material may be based on a chemical reaction of said detecting material.

The chemical reaction may be reversible.

A change from a first colour or a first or an initial visual state to or between a second colour or a second or a subsequent visual state of said detecting material may be visually observable.

The indicia may provide for a greater contrast of colour or greater visibility or greater visual contrast state with respect to said detecting material to indicate either of a detected greater or lesser parameter of fluid.

The parameter of fluid may be of a gas, and may be one or more of a gas temperature, a gas humidity content, a gas composition.

The indicia may provide for a greater contrast of colour or greater visibility or greater visual contrast state with respect to said detecting material to indicate either of a detected greater or lesser concentration of carbon dioxide relative to atmospheric carbon dioxide concentration.

The indicia may provide for a greater contrast of colour or greater visibility or greater visual contrast state with respect to said detecting material to indicate either of a detected greater or lesser concentration of one or more detected gases forming the gas composition.

The detected gases may be one or more of: carbon dioxide, oxygen, nitrogen, argon or any other gas normally present in air or inhalation/exhalation gases.

The indicia may provide for:

i. a first contrast state or a first or an initial visual state with respect to said detecting material when carbon dioxide concentration is the same or less than an atmospheric concentration of carbon dioxide, and/or

ii. a second contrast state or a second or a subsequent visual state with respect to said detecting material when carbon dioxide concentration is the greater than an atmospheric concentration of carbon dioxide.

The indicia may provide for:
a first contrast state or a first or an initial visual state with respect to said detecting material when a detected gas parameter is the same or less than a detected gas parameter of atmospheric gases, and/or
a second contrast state or a second or a subsequent visual state with respect to said detecting material when a detected gas parameter is greater than a detected gas parameter of atmospheric gases.

A first and a second indicia contrast states may be degrees of visibility.

A second indicia contrast state may be of a greater visibility or observability (i.e. appears more easily visible or optically detectable) with respect to said detecting material.

A second indicia contrast state may be significantly more visible than first indicia contrast state.

A first indicia contrast state may be when said detecting material is a first colour.

A first indicia contrast state may be when said detecting material is exposed to gas containing or comprising up to about 0.03% or 0.04% carbon dioxide (atmospheric).

A first indicia contrast state may be when carbon dioxide concentration is substantially the same as atmospheric air.

A change from a first indicia contrast state to a second indicia contrast state may be based on a chemical reaction of said detecting material.

A change from a first indicia contrast state to a second indicia contrast state may be a visually or an optically observable change in contrast (e.g. optically observable by a person).

At least one indicia may be an icon, optionally said icon resembles a pair of lungs (such as human lungs), or wind, or a heart or a graphical smiley face or other positive display of a detected carbon dioxide concentration.

The at least one indicia may be printed or mounted directly upon said component.

At least one indicia may be printed or mounted directly upon said detecting material.

The detector material may visually surround said indicia.

The detector material may visually border more than one edge or perimeter portion of said indicia.

The component may be one or more of: the detecting material, a breath indicator cap or another separate component of a breath indicator assembly.

The one or more gas flow port(s) may be one or more apertures provided as a vent hole or vent holes.

The one or more gas flow ports or vents may control the rate of gas flow or venting or allowed leak from said breath indicator.

The one or more gas flow ports may be sized to provide for a controlled vent or a pre-determined gas flow rate of gas.

A gas flow or vent rate may be about 1 litre/minute.

A gas flow port may be an aperture of a diameter of about 0 mm to about 1 mm, or about 0.7 mm.

The one or more gas flow ports may be provided through a portion of the elongate body that is provided in fluid (or gas) communication with a gas flow to be sampled or sensed, said one or more gas flow ports being downstream of a gas sampling end of said elongate body and/or downstream of said sensor or detector portion.

The one or more gas flow ports may be located substantially adjacent or about a stopping flange portion of the elongate body.

The stopping flange may be an enlarged region or portion of said elongate body.

The stopping flange may be a region or portion of said elongate body that is engageable with a portion of a device to which said indicator is attachable or engageable, to provide for a located position of said elongate body relative to a said device or to provide for a maximum or a predetermined length (or depth) of insertion of said elongate body into a device.

A length of the body may be about 40 mm.

A length of height of a cap may be about 5 mm.

The one or more gas flow ports may be substantially circular.

There may be a single gas flow port.

The indicator may further comprise: a body comprising of a gas sampling portion and a gas sensing portion or detector portion, said gas sampling portion being provided in fluid communication with said gas sensing portion or detector portion, wherein a gas detector is housed within said gas sensing portion, the gas detector comprising a material providing for a visually detectable change in colour in response to a concentration of a gas to be detected, and wherein a pre-determined indicia is provided directly upon said gas detector.

The elongate body may further comprise: a gas sampling portion and a gas sensing portion or detector portion, said gas sampling portion being provided in fluid communication with said gas sensing portion or detector portion, wherein a gas detecting material is housed within said gas sensing portion, the gas detecting material providing for a visually detectable change in colour or visual contrast state in response to a gas parameter, such as a concentration of a gas, to be detected, and wherein a pre-determined indicia is provided on or adjacent to (optionally directly upon) said gas detector.

The pre-determined indicia may be of a pre-determined shape and/or colour or visual state.

The indicia may be of a shape resembling an icon.

The icon may be indicative of breathing, lungs, wind, a heart, a graphical smiley face or other positive display of a detected carbon dioxide concentration or another visual cue.

The indicia may be directly imprinted or printed or laid upon said gas detector, or provided as a separate component which is to be directly located or laid upon said gas detector.

The gas detecting material may be a sensor, said sensor located to be exposed to a gas flow through or about said body.

The body may be an elongate body.

The elongate body may taper from a relatively smaller outer diameter substantially at or toward said gas sampling portion to a relatively larger outer diameter substantially at or toward said gas sensing portion.

The gas sampling portion may comprise a lead-in portion.

The lead-in portion may be a region of said elongate body being of a substantially narrowed (or more narrowed) outer diameter, or said lead-in portion is a tip or an end of said elongate body comprising of a chamfered edge or substantially radially inward curvature so as to assist with access or initial insertion of said gas sampling end into a device, such through as a duck-bill valve of a device.

A substantially tapered configuration of said body (such as an elongate body of said indicator) may extend from at or substantially toward said gas sampling portion toward said gas sensing portion, or detector portion, about one quarter, or about one third, or about one half, two-thirds, three-quarters, or a whole of, the length of said elongate body.

A cap or the cap may be provided as a housing to said gas detecting material or sensor or gas detector or detector portion. In another configuration, the indicia may be printed or otherwise applied to the cap.

The cap may be removable or detachable from said body.

The body may comprise a gas sampling portion and a gas sensing portion or detector portion, said gas sampling portion being provided in fluid communication with said gas sensing portion or detector portion via an elongate body, said gas sensing portion or detector portion housing a gas detector, the gas detector being of a material providing for a visually detectable change in colour or contrast state or visual state in response to a gas parameter, such as a concentration of a gas, to be detected, said gas sensing portion or detector portion being distal from said gas sampling portion, and wherein said elongate body providing for said gas sampling portion distal of said gas sensing end or detector portion, and wherein said elongate member is of a substantially tapered configuration, tapered from a relatively smaller outer diameter at or substantially toward said gas sampling end to a relatively larger outer diameter as said elongate body progresses toward said gas sensing portion or detector portion.

The gas sampling end may comprise a lead-in portion.

The lead-in portion may be a region of said elongate body being of a substantially narrowed (or more narrowed) external diameter, or said lead-in portion is a tip or end of said elongate body comprising of a chamfered edge or substantially radially inward curvature so as to assist with access or initial insertion of said gas sampling end into a device, such through as a duck-bill valve of a device.

The substantially tapered configuration may extend from at or substantially toward said gas sampling portion toward said gas sensing portion, or detector portion, about one quarter, or about one third, or about one half, two-thirds, three-quarters, or a whole of, the length of said elongate body.

A cap may be provided as a housing to said gas detector.

The cap may be removable attachable to said gas sensing portion or detector portion.

In a fourth aspect there is provided a breath indicator for providing an indication of an inhalation and/or exhalation state of a user, said breath indicator comprising:

a body comprising a gas sampling portion and a gas sensing portion or detector portion, said gas sampling portion being provided in fluid communication with said gas sensing portion or detector portion, wherein a gas detector is housed within said gas sensing portion, the gas detector comprising a material providing for a visually detectable change in colour or contrast or visual state in response to a gas parameter, such as a concentration of a gas, to be detected, and wherein a pre-determined indicia is provided on or adjacent to (optionally directly upon) said gas detector.

The pre-determined indicia may be of a pre-determined shape and/or colour.

The indicia may be of a shape resembling an icon.

The icon may be indicative of breathing, lungs, wind, a heart, a graphical smiley face or other positive display of a detected carbon dioxide concentration or another visual cue.

The indicia may be directly imprinted or printed or laid upon said gas detector, or is provided as a separate component which is to be directly located or laid upon said gas detector when provided as a separate component from said gas detector.

The gas detecting material may be a sensor, said sensor to be positioned or located to be exposed to a gas flow through or about said body.

The body may be an elongate body.

The elongate body may taper from a relatively smaller outer diameter substantially at or toward said gas sampling portion to a relatively larger outer diameter substantially at or toward said gas sensing portion or detector portion.

The gas sampling portion may comprise a lead-in portion.

The lead-in portion may be a region of said elongate body being of a substantially narrowed (or more narrowed) outer diameter, or said lead-in portion is a tip or an end of said elongate body comprising of a chamfered edge or substantially radially inward curvature so as to assist with access or initial insertion of said gas sampling end into a device, such through as a duck-bill valve of a device.

A substantially tapered configuration of said body (such as an elongate body of said indicator) may extend from at or substantially toward said gas sampling portion toward said gas sensing portion, or detector portion, about one quarter, or about one third, or about one half, two-thirds, three-quarters, or a whole of, the length of said elongate body.

A cap or the cap may be provided as a housing to said gas detecting material or sensor or gas detector or detector portion. In another configuration, the indicia may be printed or otherwise applied to the cap.

The cap may be removable or detachable from said body.

The material may be a detecting material capable of changing colour from a first colour, or a first contrast state or a first or an initial visual state, to a second colour, or a second contrast state or a second or a subsequent visual state, according to inhalation and exhalation, said detecting material housed within said elongate body, wherein said elongate body comprises a first port configured for receipt of a user's inhalation and exhalation gases and at least a second port to provide for a venting of said gases from said body, and a component comprising at least one indicia changeable as to contrast according to a change in colour or visual state of said detecting material with said user's inhalation and exhalation gases.

The detecting material may detect a parameter of a gas. The parameter may be one or more of: a gas temperature, a gas humidity content, a gas composition.

The detecting material may be a carbon dioxide detecting material or detector.

The detecting material may be of a first colour or a first or an initial visual state when a carbon dioxide concentration is about the same or substantially similar to atmospheric air.

The detecting material may be of a first colour or a first or an initial visual state when exposed to gas containing or comprising up to about 0.03% or about 0.04% carbon dioxide (atmospheric).

The detecting material may be of a second or a different colour or a second or a subsequent visual state when carbon dioxide concentration is greater than atmospheric air.

The detecting material may be of a second colour or a second or a subsequent visual state when exposed to gas containing or comprising about 2.5% or greater of a carbon dioxide concentration. A first colour or a first or an initial visual state of said detecting material may be one of: blue or purple.

A second colour or a second or a subsequent visual state of said detecting material may be one of: yellow or orange.

A change from a first colour or a first or an initial visual state to a second colour or a second or a subsequent visual state of said detecting material may be based on a chemical reaction of said detecting material.

The chemical reaction may be reversible.

A change from a first colour or a first or an initial visual state to a second colour or a second or a subsequent visual state of said detecting material may be visually observable.

The indicia may provide for a greater contrast of colour or greater visibility or greater visual contrast state with respect to said detecting material to indicate either of a detected greater or lesser parameter of fluid.

The parameter of fluid may be of a gas, and may be one or more of a gas temperature, a gas humidity content, a gas composition.

The indicia may provide for a greater contrast of colour or greater visibility or greater visual contrast state with respect to said detecting material to indicate either of a detected greater or lesser concentration of carbon dioxide relative to atmospheric carbon dioxide concentration.

The indicia may provide for a greater contrast of colour or greater visibility or greater visual contrast state with respect to said detecting material to indicate either of a detected greater or lesser concentration of one or more detected gases forming the gas composition.

The detected gases may be one or more of: carbon dioxide, oxygen, nitrogen, argon or any other gas normally present in air or inhalation/exhalation gases.

The indicia may provide for:

a first contrast state or a first or an initial visual state with respect to said detecting material when carbon dioxide concentration is the same or less than an atmospheric concentration of carbon dioxide, and/or

a second contrast state or a second or a subsequent visual state with respect to said detecting material when carbon dioxide concentration is the greater than an atmospheric concentration of carbon dioxide.

The indicia may provide for:
a first contrast state or a first or an initial visual state with respect to said detecting material when a detected gas parameter is the same or less than a detected gas parameter of atmospheric gases, and/or

a second contrast state or a second or a subsequent visual state with respect to said detecting material when a detected gas parameter is greater than a detected gas parameter of atmospheric gases.

A first and a second indicia contrast states may be degrees of visibility.

A second indicia contrast state may be of a greater visibility or observability (i.e. appears more easily visible or optically detectable) with respect to said detecting material.

A second indicia contrast state may be significantly more visible than first indicia contrast state.

A first indicia contrast state may be when said detecting material is a first colour.

A first indicia contrast state may be when said detecting material is exposed to gas containing or comprising up to about 0.03% or about 0.04% carbon dioxide (atmospheric).

A first indicia contrast state may be when carbon dioxide concentration is substantially the same as atmospheric air.

A change from a first indicia contrast state to a second indicia contrast state may be based on a chemical reaction of said detecting material.

A change from a first indicia contrast state to a second indicia contrast state may be a visually or an optically observable change in contrast (e.g. optically observable by a person).

At least one indicia may be an icon, optionally said icon resembles a pair of lungs (such as human lungs), or wind, or a heart or a graphical smiley face or other positive display of a detected carbon dioxide concentration.

The at least one indicia may be printed or mounted directly upon said component.

At least one indicia may be printed or mounted directly upon said detecting material.

The detector material may visually surround said indicia.

The detector material may visually border more than one edge or perimeter portion of said indicia.

The detector material may visually fill or encompass an area of the component that is not said indicia.

The component may be one or more of: the detecting material, a breath indicator cap or another separate component of a breath indicator assembly.

The breath indicator may further comprise of said body in the form of an elongate body, a cap, a sensor or detector portion comprising of said detecting material, said sensor or detector portion positioned to be exposed to a gas flow through said elongate body into a region housed by said cap.

The cap may include a radially inwardly extending projection portion for holding or retaining the cap and the elongate body together in an engaged configuration.

The projection portion may engage with an exterior portion of said elongate body.

The projection portion may extend circumferentially inwardly around the cap.

The cap may be receivable of an end of said elongate body, said cap configured to be retained upon said end of said elongate body.

The end of said elongate body may be an end configured to receive or locate said sensor.

The end of said elongate body may be configured to provide a platform to receive or locate said sensor.

The end of said elongate body and said cap, when in said engaged configuration may provide a housing for said sensor.

The elongate body may comprise of a hollow passageway extending between ends of said elongate body, a first end of said elongate body provided as a gas sampling end and a second end of said elongate body provided as a region for receipt or locating of said sensor.

The second end of said elongate body may comprise a platform region upon which said sensor is to be received or located, said hollow passageway terminating into said platform region.

A detecting material may be received or located in a void space provided between an end of said elongate body and an internal wall surface of said cap (when said cap is in-situ or in an engaged or an assembled configuration with said elongate body).

A component with at least one indicia may be printed or mounted directly thereon is received or located in a said void space between an end of said elongate body and an underside or an internal wall surface side of said cap (when said cap is in-situ or in an engaged or an assembled configuration with said elongate body).

The component may be one or more of: the sensor, the detecting material, the cap, or the component is provided as a separate item from each of the aforementioned items (being the sensor, the detecting material, the cap or the component).

The cap may be formed, at least in part, of an optically transparent material.

The cap (optionally a shell or a lid portion of said cap) may provide for an optical magnification capability of said sensor and/or said indicia to be located beneath said cap (or beneath the shell or lid portion of said cap).

The elongate body may comprise of an open end or a gas inlet and a flow path or passageway or conduit extending from said open end or the gas inlet to said sensor or detector portion.

The open end or gas inlet may be provided as a gas sampling or gas receiving end of said elongate body.

The flow path or passageway or conduit may provide for fluid (i.e. gas) communication between a gas sampling end of said elongate body and a gas sensing end (or the sensor end) or detector portion of said elongate body.

The elongate body may comprise of a terminal end of a flow path or passageway or conduit extending from an open end or a gas inlet into said elongate body, said terminal end terminated by said cap (when said cap is in-situ or in an engaged or an assembled configuration with said elongate body).

The terminal end may comprise of at least one gas flow port as a vent or outlet to atmosphere for discharging of a gas flow directed from a gas sampling end of said elongate body through said terminal end to be discharged to atmosphere.

The one or more gas flow ports may be one or more apertures provided as a vent hole or vent holes.

The one or more gas flow ports or vents may control the rate of gas flow or venting or allowed leak from said breath indicator.

The one or more gas flow ports may be sized to provide for a controlled vent or a pre-determined gas flow rate of gas.

A gas flow or vent rate may be about 1 litre/minute.

A gas flow port may be an aperture of a diameter of about 0 mm to about 1 mm, or about 0.7 mm.

The one or more gas flow ports are provided through a portion of the elongate body that is provided in fluid (or gas) communication with a gas flow to be sampled or sensed, said one or more gas flow ports being downstream of a gas sampling end of said elongate body and/or downstream of said sensor or detector portion.

The one or more gas flow ports may be located substantially adjacent or about a stopping flange portion of the elongate body.

The stopping flange may be an enlarged region or portion of said elongate body.

The stopping flange may be a region or portion of said elongate body that is engageable with a portion of a device to which said indicator is attachable or engageable, to provide for a located position of said elongate body relative to a said device or to provide for a maximum or a predetermined length (or depth) of insertion of said elongate body into a device.

A length of the body may be about 40 mm.

A length of height of a cap may be about 5 mm.

The one or more gas flow ports may be substantially circular shaped aperture(s).

There may be a single (one or sole) gas flow port.

The gas sampling portion may be provided in fluid communication with said gas sensing portion or detector portion via said body in the form of an elongate body, said gas sensing portion or detector portion housing said gas detector, said gas sensing portion or detector portion being distal from said gas sampling portion, and wherein said elongate body providing for said gas sampling portion distal of said gas sensing end or detector portion, and wherein said elongate member is of a substantially tapered configuration, tapered from a relatively smaller outer diameter at or substantially toward said gas sampling end to a relatively larger outer diameter as said elongate body progresses toward said gas sensing portion or detector portion.

The gas sampling end may comprise a lead-in portion.

The lead-in portion may be a region of said elongate body being of a substantially narrowed (or more narrowed) external diameter, or said lead-in portion is a tip or end of said elongate body comprising of a chamfered edge or substantially radially inward curvature so as to assist with access or initial insertion of said gas sampling end into a device, such through as a duck-bill valve of a device.

The substantially tapered configuration may extend from at or substantially toward said gas sampling portion toward said gas sensing portion, or detector portion, about one quarter, or about one third, or about one half, two-thirds, three-quarters, or a whole of, the length of said elongate body.

A cap may be provided as a housing to said gas detector.

The cap may be removably attachable to said gas sensing portion or detector portion.

In a fifth aspect, there is provided a breath indicator for providing an indication of an inhalation and/or exhalation state of a user, said breath indicator comprising:

a body comprising a gas sampling portion and a gas sensing portion or detector portion,

said gas sampling portion being provided in fluid communication with said gas sensing portion or detector portion via an elongate body,

said gas sensing portion or detector portion housing a gas detector, the gas detector being of a material providing for a visually detectable change in colour or contrast state in response to a gas parameter, such as a concentration of a gas, to be detected, said gas sensing portion or detector portion being distal from said gas sampling portion,

and wherein said elongate body providing for said gas sampling portion distal of said gas sensing end or detector portion, and wherein said elongate member is of a substantially tapered configuration, tapered from a relatively smaller outer diameter at or substantially toward said gas sampling end to a relatively larger outer diameter as said elongate body progresses toward said gas sensing portion or detector portion.

The gas sampling end may comprise a lead-in portion.

The lead-in portion may be a region of said elongate body being of a substantially narrowed (or more narrowed) external diameter, or said lead-in portion is a tip or end of said elongate body comprising of a chamfered edge or substantially radially inward curvature so as to assist with access or initial insertion of said gas sampling end into a device, such through as a duck-bill valve of a device.

The substantially tapered configuration may extend from at or substantially toward said gas sampling portion toward said gas sensing portion, or detector portion, about one quarter, or about one third, or about one half, two-thirds, three-quarters, or a whole of, the length of said elongate body.

A cap may be provided as a housing to said gas detector.

The cap may be removably attachable to said gas sensing portion or detector portion.

The indictor may the indicator as described in any one or more of the aforementioned first, second, third or fourth aspects.

The breath indicator may include or comprise one or more gas flow ports for venting a gas from said indicator to atmosphere.

The one or more gas flow ports or vents may be one or more apertures provided as a vent hole or holes.

The one or more gas flow ports or vents may control the rate of gas flow or venting or allowed leak from said breath indicator.

The one or more gas flow ports may be sized to provide for a controlled vent or gas flow rate of gas.

A gas flow or vent rate may be about 1 litre/minute.

A gas flow port may be an aperture of a diameter of about 0 mm to about 1 mm, or about 0.7 mm.

The one or more gas flow ports may be provided within or through portion of the elongate body that is provided in fluid (or gas) communication with a gas flow to be sampled or sensed, said one or more gas flow ports being downstream of a gas sampling end of said elongate body or downstream of a sensor (optionally said sensor comprising of a detecting material, said sensor positioned or located to be exposed to a gas flow through or about said elongate body and/or said cap).

The one or more gas flow ports may be substantially adjacent or located about a stopping flange portion of the elongate body.

The stopping flange may be an enlarged region or portion of said elongate body.

The stopping flange may be a region or portion of said elongate body that is engageable with a portion of a device to which said indicator is attachable or engageable, to provide for a located position of said elongate body relative to a said device or to provide for a maximum or predetermined length (or depth) of insertion of said elongate body into a device.

A length of the body may be about 40 mm.

A length of height of a cap may be about 5 mm.

The one or more gas flow ports may be circular aperture(s).

There may be a single (one or sole) gas flow port or vent.

In a sixth aspect, there is provided a breath indicator for providing an indication of a breathing condition or state or a user, such as an inhalation and/or an exhalation condition or state of a user, said breath indicator comprising:

the breath indicator as claimed in any one of the aforementioned aspects, being the first aspect or the second aspect or the third aspect or the fourth aspect or the fifth aspect,

wherein said breath indicator includes one or more gas flow ports for venting a gas from said indicator to atmosphere.

The one or more gas flow ports or vents may be one or more apertures provided as a vent hole or holes.

The one or more gas flow ports or vents may control the rate of gas flow or venting or allowed leak from said breath indicator.

The one or more gas flow ports may be sized to provide for a controlled vent or gas flow rate of gas.

A gas flow or vent rate may be about 1 litre/minute.

A gas flow port may be an aperture of a diameter of about 0 mm to about 1 mm, or about 0.7 mm.

The one or more gas flow ports may be provided within or through portion of the elongate body that is provided in fluid (or gas) communication with a gas flow to be sampled or sensed, said one or more gas flow ports being downstream of a gas sampling end of said elongate body or downstream of a sensor (optionally said sensor comprising of a detecting material, said sensor positioned or located to be exposed to a gas flow through or about said elongate body and/or said cap).

The one or more gas flow ports may be substantially adjacent or located about a stopping flange portion of the elongate body.

The stopping flange may be an enlarged region or portion of said elongate body.

The stopping flange may be a region or portion of said elongate body that is engageable with a portion of a device to which said indicator is attachable or engageable, to provide for a located position of said elongate body relative to a said device or to provide for a maximum or predetermined length (or depth) of insertion of said elongate body into a device.

A length of the body may be about 40 mm.

A length of height of a cap may be about 5 mm.

The one or more gas flow ports may be circular aperture(s).

There may be a single (one or sole) gas flow port or vent.

In a seventh aspect the invention may be said to broadly consist of breath indicator receivable by a part of a breathing assistance apparatus that supplies gas to a user or patient, the indicator comprising:

an elongate body having a gas sampling end and an attachment end, the attachment end adapted to attach to a part of a breathing assistance apparatus and for locating the gas sampling end, and the gas sampling end to be located in a region where gas from the patient is to be exhaled, the gas sampling end being in communication with a sensor or detector portion comprising a detector or detecting material changeable between a first visual indicator state, or an initial visual state, relating to an inhalation phase of the patient, and a second visual indicator state, or subsequent visual state, relating to an exhalation phase of the patient, and wherein the detector or detecting material is capable of changing between the visual indicator states at a sufficient rate to substantially correspond with the inhalation and exhalation phases of the user or patient, and wherein said detector or detecting material has associated therewith one or more of:
a component comprising at least one indicia changeable as to contrast or visibility or visual state, according to a change in colour or visual state or response of said detector or detecting material with said user's inhalation and exhalation gases;
a pre-determined indicia or reference marking or a reference material provided directly upon said detector or detecting material.

The detecting material may detect a parameter of a gas. The parameter may be one or more of: a gas temperature, a gas humidity content, a gas composition.

The detecting material is CO₂ detecting material.

The sensor comprises CO₂ detecting material.

The detecting material changes from a first visual indicator state or a first or an initial visual state to a second indicator state or a second or a subsequent visual state when exposed to gas having CO₂ concentration greater than that normally found in atmospheric air.

The detecting material changes from a second visual indicator state or a second or a subsequent visual state to a first indicator state or a first or an initial visual state when exposed to gas having CO₂ concentration the same or substantially similar to those normally found in atmospheric air.

The detecting material changes from the first visual indicator state (a first colour) or a first or an initial visual state to a second visual indicator state (a second colour) or a second or a subsequent visual state when exposed to gas having a concentration by volume of at least about 2.5% or more CO₂.

The visual indicator state of the detecting material can be optically or visually observed by a user or patient of a breathing assistance apparatus for a patient.

In-use, end-tidal or breath-by-breath CO₂ present within gas exhaled by a user or patient is optically or visually observable by a user by change in visual indictor state of the detecting material.

At least a part of the attachment end remains external of a part of a breathing assistance apparatus to which the breath indicator is attached.

The attachment end is provided substantially perpendicular to the elongate body.

The attachment end is disk-shaped.

The attachment end is formed as a handle portion including a handle flange extending outwards perpendicularly from the end of the attachment end.

The breath indicator further comprises a stopping flange located partway along said elongate body and extending substantially perpendicularly from said body, said flange located between said attachment end and said sampling end.

The breath indicator is receivable by a patient interface.

The patient interface comprises any one or combination of the following: a face mask, an oral mask, an oro-nasal mask, a nasal mask, one or a pair of nasal prongs, an endotracheal tube, a T-piece resuscitator apparatus, gas flow regulator or gas pressure regulator associated with any one or more of these.

The breath indicator is receivable by a face mask.

The breath indicator is receivable by a T-piece breathing assistance apparatus.

The exterior surface of the elongate body is substantially smooth, or otherwise shaped, such that, in-use the elongate body does not increase the resistance to flow of gas through a part of a breathing assistance apparatus to which the indicator is attached.

The elongate body may have an outer diameter of about 1 mm to about 5 mm, and a length of about 30 mm to about 60 mm.

The gas sampling end of the elongate body comprises a gas inlet, the inlet being in fluid communication with the sensor via a passageway.

A pressure difference exists between the gas sampling end and the attachment end, or detector portion, when in-use.

The sensor or detector portion comprising the detecting material is provided in-line with the passageway.

The passageway extends from the gas sampling end of the elongate body to the sensor or detector portion.

The passageway provides for a gas flow path extending from the breath indicator gas inlet to one or more ports, said ports providing for breath indicator gas outlets, the port or ports in fluid communication with the surrounding atmosphere external of the resuscitation system, the sensor or detecting material positionable for contact with the gas flow path through the passageway.

The sensor with detecting material is located in a region adjacent the attachment end, and wherein the attachment end, or at least a part of the attachment end, is formed of a visually transparent material allowing a user or patient to visually detect changes between the first and second visual indicator states of the detector material.

The attachment end houses the sensor.

The attachment end comprises a housing locating the sensor.

At least a part of the attachment end (or housing) is formed of a visually transparent material, such that in-use, a user is able to visually detect changes between the first or an initial visual indicator or visual state and a second visual indicator or a subsequent visual indicator or visual state of the detector material.

The elongate body has a length sufficient to allow the gas sampling end to be located in a region where gas from the patient is to be exhaled, and where the attachment end is located in a position attached to a part of a breathing assistance apparatus.

The sampling end and sensor are substantially adjacent, or are the same part of region of the elongate body.

The sensor is formed as a hollow section of the sampling end, the hollow section filled with an indicator dye, the wall or shell of said hollow section having pores to allow gases to pass through the wall or shell and contact the indicator dye, the wall or shell substantially clear or translucent to allow a user to view the colour of the dye through the wall or shell.

The detecting material is a layer of indicator dye applied to the outside surface of the sampling end, the detecting material applied to at least part of the area or region of the elongate body near the sampling end.

The sampling end is at least partly (or wholly) formed from detecting material and attached or connected to the remainder of the elongate body.

The detecting material is applied to the entire area of said elongate body.

The detecting material is a layer of material infused with a CO₂ detecting material and applied to the outside surface of the sampling end.

The elongate body includes a plurality of apertures.

The apertures are arranged in a substantially honeycomb pattern.

The apertures are circular, oval, square, rectangular or triangular-shaped.

The apertures are positioned adjacent to the attachment end of the elongate body.

The apertures are positioned about 1 mm to about 15 mm along the elongate body from the attachment end.

The apertures are positioned about 7 mm to about 10 mm along the elongate body from the attachment end.

The apertures are positioned adjacent to the sensor.

The apertures are positioned about 1 mm to about 15 mm along the elongate body from the sampling end.

The apertures are positioned about 7 mm to about 10 mm along the elongate body from the sampling end.

In-use the elongate body is inserted into a breathing assistance apparatus that supplies gas to a user or patient, such that the sampling end is located proximate to the mouth and nose of the user or patient and such that the apertures are positioned in a flow of gas from an inlet to the breathing assistance apparatus, and wherein, in-use, gas from the inlet flow through the apertures draws gas exhaled by a user or patient toward the sensor, such that exhaled gas can come into contact with the detecting material.

In use the attachment end, when attached to a part of a resuscitator system, aligns the elongate body with apertures with flow of gas from the inlet to the breathing assistance apparatus.

The breathing assistance apparatus is, or forms a part of, a resuscitator system.

Accordingly, in an eighth aspect the invention can be said to broadly consist of a breath indicator as part of a resuscitator system that supplies gases to a user or patient via a patient interface (such as a face mask or other interface), comprising:

an elongate body having a sensing end and an attachment end, said sensing end comprising a detector or detecting material which changes between two visual indicator states, a first visual indicator state relating to an inhalation phase of the patient or user, and a second visual indicator state relating to an exhalation phase, the detector or detecting material capable of changing between the visual indicator states at a sufficient rate to substantially correspond to inhalation and exhalation changes of the patient to indicate inhalation and exhalation, said elongate body adapted so that in use, said sensing end can be located proximate to the mouth and/or nose of said patient, and wherein said detector or detecting material has associated therewith one or more of:
a component comprising at least one indicia changeable as to contrast or visibility or visual state according to a change in colour or visual state or response of said detector or detecting material with said user's inhalation and exhalation gases;
a pre-determined indicia or reference marking or a reference material provided directly upon said detector or detecting material.

The sensing end comprises CO₂ detecting material which can change colour from a base colour being the first visual indicator state to a detection colour being the second visual indicator state when exposed to gases which have CO₂ concentration greater than that normally found in atmospheric air, and back to the base colour when exposed to gases which have CO₂ concentrations the same or similar to those found in atmospheric air, so as to allow a medical professional viewing said first end to detect end-tidal or breath-by-breath CO₂ present within said breathing assistance apparatus in use.

The breath indicator further comprises a stopping flange located partway along said elongate body and extending substantially perpendicularly from said body, said flange located between said attachment end and said sensing end.

The detecting material changes colour from a base colour to a detecting colour when exposed to gases having a concentration by volume of at least 2.5% or more CO₂.

The attachment end is formed as a handle portion that includes a handle flange extending outwards perpendicularly from the end of said attachment end.

The sensing end is formed as a hollow section, said hollow section filled with an indicator dye, the wall or shell of said hollow section having pores to allow gases to pass through said wall or shell and contact said indicator dye, said wall or shell substantially clear or translucent to allow a user to view the colour of the dye through said wall or shell.

The detecting material is a layer of indicator dye applied to the outside surface of said sensing end, said detecting material applied to at least part of the area of the elongate body near said sensing end.

Alternatively, wherein said sensing end is at least partly and may be wholly formed from detecting material and attached or connected to the remainder of said elongate body.

As a further alternative said detecting material is applied to the entire area of said elongate body.

The detecting material is a layer of material infused with a CO₂ detecting material and applied to the outside surface of said sensing end.

The indicator may have an outer diameter between 1 mm and 5 mm, said indicator also having a length of about 30 mm to about 60 mm, and said elongate body being substantially smooth to not increase the resistance to flow.

The elongate indicator includes a plurality of apertures in said indicator

The apertures may be arranged in a honeycomb pattern.

The apertures may be any shape including circular, oval, square, rectangular or triangular.

The apertures are positioned adjacent said attachment end of said indicator.

The apertures are positioned about 1 mm to about 15 mm away and may be about 7 mm to about 10 mm away from said attachment end along said elongate body.

Alternatively, the apertures are positioned adjacent to said sensing end.

In the alternative, the apertures are positioned anywhere from about 1 mm to about 15 mm away, and may be about 7 mm to about 10 mm away from said sensing end along said elongate body.

In-use said indicator is inserted into a breathing assistance apparatus that supplies gases to a user or patient, such that said sensing end is located proximate to the mouth and nose of the user or patient and such that said apertures are positioned in a flow of gases from an inlet of the breathing assistance apparatus, wherein use gases from the inlet flow through said apertures to help draw gases exhaled by a user or patient toward said indicator such that exhaled gases can come into contact with said detecting material.

The apertures are further positioned to reduce the resistance of the flow of gases from the inlet.

In-use the locking feature positions the apertures such that the apertures are aligned with the flow of gases from the inlet and/or such that said apertures are positioned to reduce the resistance of the flow of gases from the inlet.

In a ninth aspect the invention can be said to broadly consist of a breathing assistance apparatus for use as part of a resuscitation system for a patient or user via a patient interface (such as a face mask or other interface, the breathing assistance apparatus comprising:

a manifold section, said manifold section hollow to define a gases space, a gases inlet passing from outside said manifold section into said gases space, said gases inlet adapted for connection to a gases conduit or similar to receive a continuous flow of gases at a pressure above atmospheric and further adapted to allow said continuous flow of gases to enter said gases space, said continuous flow of gases forming a gases stream, a gases outlet from said manifold, in use said gases stream passing through said outlet to a user or patient, a device inlet on said manifold section adapted to allow items to pass into said gases space from outside said manifold section, a breath indicator formed from an elongate body having a sensing end and an attachment end, said sensing end comprising detecting material which changes between two visual indicator states, a first visual indicator state relating to an inhalation phase of the user or patient, a second visual indicator state relating to an exhalation phase, the detecting material capable of changing between the visual indicator states at a sufficient rate to substantially correspond to inhalation and exhalation changes of the user or patient to indicate inhalation and exhalation, said breath indicator formed separately from said manifold, said breath indicator disposed into said device inlet so that said sensing end protrudes into said gases space and said sensing end is substantially proximate to at least the gases outlet, and wherein said detector or detecting material has associated therewith one or more of:
a component comprising at least one indicia changeable as to contrast or visibility according to a change in colour or visual state or response of said detector or detecting material with said user's inhalation and exhalation gases;
a pre-determined indicia or reference marking or a reference material provided directly upon said detector or detecting material.

In-use the user or patient exhales into the outlet and the gases space of the manifold and said breath indicator and said manifold section are mutually sized so that said sensing end in use can be located at or close to an opening of the outlet, such that the sensing end is in the gases path of exhaled gases from the user or patient.

At least part of said manifold section is translucent.

At least that part of said manifold closest to said user or patient in use is translucent.

The breath indicator further comprises a stopping flange located partway along said elongate body and extending substantially perpendicularly from said body, said stopping flange located between said attachment end and said sensing end, in use said stopping flange contacting and abutting the perimeter of said device inlet and preventing further travel of said breath indicator through said device inlet.

The breath indicator may be characterised by the sensing end comprising a CO₂ detecting material which can change colour or visual state from a base colour or base visual state being the first or an initial visual indicator state to a detection colour being the second or a subsequent visual indicator state when exposed to a gases parameter, such as which have CO₂ concentration, greater than that normally found in atmospheric air, and back to the base colour or base visual state when exposed to gases which a gases parameter, such as which have CO₂ concentrations, the same or similar to those found in atmospheric air, so as to allow a medical professional viewing said first end to detect end-tidal or breath-by-breath, such as CO₂ present, within said breathing assistance apparatus in use

The detecting material changes colour from a base colour or base visual state to a detecting colour or detecting visual state when exposed to gases having a gases parameter substantially corresponding with a gases parameter corresponding with exhalation gases.

The detecting material changes colour from a detecting colour or detecting visual state base colour or base visual state to a base colour or base visual state when exposed to gases having a gases parameter substantially corresponding with a gases parameter corresponding with inhalation gases.

The detecting material changes colour from a base colour or base visual state to a detecting colour or detecting visual state when exposed to gases having a concentration by volume of at least 2.5% or more CO₂.

The attachment end is formed as a handle portion that includes a handle flange extending outwards perpendicularly from the end of said attachment end.

A portion of the body of said breath indicator adjacent to said stopping flange and on the side opposite to said sensing end is formed as a handle portion.

The sensing end is formed as a hollow section, said hollow section filled with an indicator dye, the wall or shell of said hollow section having pores to allow gases to pass through said wall or shell and contact said indicator dye, said wall or shell substantially clear or translucent to allow a user to view the colour of the dye through said wall or shell.

The CO₂ detecting material is a layer of indicator dye applied to the outside surface of said sensing end.

The detecting material is a layer of indicator dye applied to the outside surface of said sensing end of the breath indicator, said detecting material applied to at least part of the area of the elongate body near said sensing end of the breath indicator.

The sensing end of the breath indicator is at least partly and may be wholly formed from detecting material and attached or connected to the remainder of said elongate body.

The detecting material is applied to the entire area of said elongate body of the breath indicator.

The sensing end of said breath indicator is at least partly and may be wholly formed from CO₂ detecting material and attached or connected to the remainder of said elongate body.

Alternatively, the CO₂ detecting material is a layer of material infused with a CO₂ detecting material and applied to the outside surface of said sensing end of the breath indicator.

A duck billed valve may be located in said device inlet, allowing the passage of items from the exterior to the interior of the manifold section and sealing to prevent the flow of gases from the interior to the exterior of said manifold section during use when no items are inserted, during the insertion of items, for the duration of the time for which the items are inserted, and as the items are removed from the manifold section.

The breath indicator may further have a locking feature, said device inlet further having a fastening feature, said locking feature corresponding with and engaging with said fastening feature in use to retain said breath indicator within said device inlet.

In one form said CO₂ detecting is applied to at least part of an inside surface of said manifold section.

The indicator has an outer diameter between 1 mm and 5 mm, said indicator also having a length of about 30 mm to about 60 mm.

The elongate indicator includes a plurality of apertures in said indicator

The apertures may be arranged in a honeycomb pattern.

The apertures may be any shape including circular, oval, square, rectangular or triangular.

The apertures are positioned close to said attachment end of said indicator.

The indicator is shaped to not increase resistance to the flow of gases through said manifold section.

In use said apertures are substantially aligned with the flow of gases into the manifold from the inlet such that said apertures are positioned in the flow of gases to help draw gases exhaled by a user or patient toward said indicator such that exhaled gases can come into contact with said detecting material.

The apertures are further positioned to reduce the resistance of the flow of gases from the inlet.

The manifold section comprising a PEEP outlet opening from manifold to said atmosphere, said PEEP outlet including an aperture opening to atmosphere, said PEEP outlet capable of being occluded and unoccluded manually or automatically, said PEEP outlet positioned substantially opposite to said inlet, gases flowing from said inlet to and out of said PEEP outlet when said PEEP outlet is unoccluded, said gases flowing through said apertures in said indicator and around said indicator, said flow of gases through said apertures causing a drop in pressure across said indicator or due to changing velocities of said gases flow, causing any exhaled gases from said user or patient to be drawn toward said indicator.

In use said locking feature positions the apertures in the flow of gases such that the apertures are positioned to reduce the effect on flow resistance of the flow of gases from the inlet.

In a tenth aspect the invention can be said to broadly consist in a breath indicator as part of a resuscitator system for a user or patient that supplies gases to a user or patient via a patient interface, comprising:

an elongate body having a sensing portion and an attachment portion, the elongate body comprising a plurality of apertures along a length of the elongate body, said sensing portion comprising detecting material which changes between two visual indicator states, a first visual indicator state relating to an inhalation phase of the patient, and a second visual indicator state relating to an exhalation phase, the detecting material capable of changing between the visual indicator states at a sufficient rate to substantially correspond to inhalation and exhalation changes of the user or patient to indicate inhalation and exhalation, wherein in-use said indicator is inserted into a breathing assistance apparatus that supplies gases to a user or a patient, such that said sensing portion is located proximate to the mouth and nose of the user or patient and such that said apertures are positioned in a flow of gases from an inlet of the breathing assistance apparatus, where in use gases from the inlet flow through said apertures to help draw gases exhaled by a user or patient toward said indicator such that exhaled gases can come into contact with said detecting material, and wherein said detector or detecting material has associated therewith one or more of:
a component comprising at least one indicia changeable as to contrast or visibility according to a change in colour or response or visual state of said detector or detecting material with said user's inhalation and exhalation gases;
a pre-determined indicia or reference marking or a reference material provided directly upon said detector or detecting material.

In an eleventh aspect the invention is said to broadly consist in a breathing assistance apparatus for use as part of a resuscitation system for a user or patient, the breathing assistance apparatus comprising:

a manifold section, said manifold section hollow to define a gases space, a gases inlet passing from outside said manifold section into said gases space, said gases inlet adapted for connection to a gases conduit or similar to receive a continuous flow of gases at a pressure above atmospheric and further adapted to allow said continuous flow of gases to enter said gases space, said continuous flow of gases forming a gases stream, a gases outlet from said manifold, in use said gases stream passing through said outlet to a user or patient, a device inlet on said manifold section adapted to allow items to pass into said gases space from outside said manifold section, a breath indicator formed from an elongate body having a gas sensing portion and an attachment portion, the elongate body comprising a plurality of apertures along a length of the elongate body, said gas sensing portion comprising detecting material which changes between two visual indicator states, a first visual indicator state relating to an inhalation phase of the user or patient, and a second visual indicator state relating to an exhalation phase, the detecting material capable of changing between the visual indicator states at a sufficient rate to substantially correspond to inhalation and exhalation changes of the user or patient to indicate inhalation and exhalation, wherein in-use said indicator is inserted into a breathing assistance apparatus that supplies gases to a user or patient, such that said sensing portion is located proximate to the mouth and nose of the user or patient and such that said apertures are positioned in a flow of gases from an inlet of the breathing assistance apparatus, where in use gases from the inlet flow through said apertures help to draw gases exhaled by a user or patient toward said indicator such that exhaled gases can come into contact with said detecting material, and wherein said detector or detecting material has associated therewith one or more of:
a component comprising at least one indicia changeable as to contrast or visibility or visual state according to a change in colour or response or visual state of said detector or detecting material with said user's inhalation and exhalation gases;
a pre-determined indicia or reference marking or a reference material provided directly upon said detector or detecting material.

In a twelfth aspect the invention is said to broadly consist in a breath indicator as part of a resuscitator system for a user or patient that supplies gases to a user or patient via a patient interface, comprising:

a body having a sensing portion and an attachment portion, said sensing portion comprising detecting material which changes between two visual indicator states, a first or initial visual indicator state or visual state relating to an inhalation phase of the user or patient, and a second or subsequent visual indicator state or visual state relating to an exhalation phase, the detecting material capable of changing between the visual indicator states or visual states at a sufficient rate to substantially correspond to inhalation and exhalation changes of the user or patient to indicate inhalation and exhalation,

where in use said indicator is inserted into a breathing assistance apparatus that supplies gases to an user or patient, such that said sensing portion is located proximate to the mouth and nose of the user or patient, such that said sensing portion is positioned in a flow of gases from an inlet of the breathing assistance apparatus, and a flow of gases exhaled by an user or patient toward said indicator such that exhaled gases can come into contact with said detecting material, and wherein said detector or detecting material has associated therewith one or more of:
a component comprising at least one indicia changeable as to contrast or visibility or visual state according to a change in colour or response or visual state of said detector or detecting material with said user's inhalation and exhalation gases;
a pre-determined indicia or reference marking or a reference material provided directly upon said detector or detecting material.

In a thirteenth aspect the invention is said to broadly consist in a breathing assistance apparatus for use as part of a resuscitation system for a user or patient, the breathing assistance apparatus comprising:

a manifold section, said manifold section hollow to define a gases space, a gases inlet passing from outside said manifold section into said gases space, said gases inlet adapted for connection to a gases conduit or similar to receive a continuous flow of gases at a pressure above atmospheric and further adapted to allow said continuous flow of gases to enter said gases space, said continuous flow of gases forming a gases stream, a gases outlet from said manifold, in use said gases stream passing through said outlet to a user or patient, a device inlet on said manifold section adapted to allow items to pass into said gases space from outside said manifold section, a breath indicator formed from a body having a sensing portion and an attachment portion, said sensing portion comprising detecting material which changes between two visual indicator states, a first or initial visual indicator state relating to an inhalation phase of the user or patient, and a second or subsequent visual indicator state relating to an exhalation phase, the detecting material capable of changing between the visual indicator states at a sufficient rate to substantially correspond to inhalation and exhalation changes of the user or patient to indicate inhalation and exhalation, wherein in-use said indicator is inserted into a breathing assistance apparatus that supplies gases to an user or patient, such that said sensing portion is located proximate to the mouth and nose of the user or patient, such that said sensing portion is positioned in a flow of gases from an inlet of the breathing assistance apparatus, and a flow of gases exhaled by an user or patient toward said indicator such that exhaled gases can come into contact with said detecting material, and wherein said detector or detecting material has associated therewith one or more of:
a component comprising at least one indicia changeable as to contrast or visibility or visual state according to a change in colour or response or visual state of said detector or detecting material with said user's inhalation and exhalation gases;
a pre-determined indicia or reference marking or a reference material provided directly upon said detector or detecting material.

In a fourteenth aspect the invention is said to broadly comprise a breath indicator for providing an indication of an inhalation and/or exhalation state of a user, said breath indicator comprising:

a body comprising a gas sampling portion configured for receipt of a user's inhalation and/or exhalation gases, and an indicator portion, said gas sampling portion in fluid communication with said indicator portion,

said indicator portion comprising at least a first region and at least a second region, the first region comprising a gas parameter detecting material capable of changing between an initial visual state and a subsequent visual state indicative of detection of a gas parameter of inhalation and/or exhalation gases of said user; and

wherein said initial visual state and/or said subsequent visual state of the first region visually contrasts with a visual state of at least said second region.

The first region may be shaped in the form of an indicium.

The second region may be shaped in the form of an indicium.

Either the first region and/or the second region may substantially surround the respective second region and/or the first region.

The second region may comprise a static or substantially unchanging visual state.

The second region may comprise a gas parameter detecting material capable of changing from an initial visual state to a subsequent visual state, said change indicative of a detected gas parameter of inhalation and/or exhalation gases of said user.

The indicator portion may comprise at least one further region, said further region comprising a static or substantially unchanging visual state.

The indicator portion may comprise at least one further region, said further region comprises a gas parameter detecting material capable of changing from an initial visual state to a subsequent visual state, said change indicative of a detected gas parameter of inhalation and/or exhalation gases of said user.

The further region may be in the form of an indicium.

One or more of the first region and/or the second region and/or a further region of said indicator portion are substantially adjacent each other, or are substantially surrounding of one or more of the first region and/or the second region and/or a further region.

The first region may be substantially adjacent to said second region.

The first region is substantially adjacent to said further region.

The second region may be substantially adjacent to said further region.

At least one of said first region or said second region, or a further region, may be provided as a background visual state.

The background visual state may be provided by a gas parameter detecting material capable of changing from changing from an initial visual state to a subsequent visual state, being indicative of a detected gas parameter of inhalation and/or exhalation gases of said user.

The background visual state may be a static or substantially unchanging visual state.

The indicium may be of a pre-determined shape and/or colour.

The indicium may be, or may resemble, an icon.

The indicium may be indicative of breathing, lungs, wind, a heart, a graphical smiley face or other positive display of a detected gas parameter or another visual cue.

The body may be an elongate body.

The elongate body may taper from a relatively smaller outer or external diameter substantially at or toward said gas sampling portion to a relatively larger outer or external diameter substantially at or toward said indicator portion.

The gas sampling portion may comprise a lead-in portion.

The lead-in portion may be at least one of (i) a region of said elongate body having a comparatively narrowed external diameter, or (ii) a tip or end of said elongate body comprising a chamfered edge or substantially radially inward curvature so as to assist with access of said gas sampling end into a device.

A substantially tapered configuration of said body may extend from said gas sampling portion toward said indicator portion about one quarter, or about one third, or about one half, two-thirds, three-quarters, or a whole of, the length of said elongate body.

The indicator portion may comprise a cap configured to substantially cover the gas parameter detecting material and complete a gas pathway between the gas sampling portion and the indicator portion.

The indicium may be imprinted, printed or laid upon said cap, or may be provided as a separate component which is locatable on said cap when provided as a separate component.

The cap may be removable or detachable from said body.

The cap may be rotatably engageable with the body.

The cap may be one or more of:
engaged to the body via one or more locking cam(s),
threadably attachable to the body,
push fit to the body.

The cap and body may be configured such that the engagement is by helical rotation.

The cap and the indicator portion may comprise one or more complementary radial ramp structures configured to provide the helical rotation.

The indicator portion may comprise at least one tab configured to engage the cap.

The cap may comprise a slot or aperture configured to accept the at least one tab of the indicator portion.

The tab may provide for a standoff feature configured to locate the cap in a predetermined position.

The predetermined position may be a height relative to the gas parameter detecting material.

The predetermined position may be a rotated location or angular position relative to the indicator portion.

The cap may be configured to substantially enclose the indicator portion.

The cap may be substantially transparent or translucent to provide for a visual identification of a changing colour and/or visual state of the gas parameter detecting material.

The indicator portion may comprise at least one port configured to vent to atmosphere.

The cap may comprise at least one port configured to vent to atmosphere.

The body may comprise a gas pathway between the gas sampling portion and the indicator portion, the gas pathway configured to expose or contact or distribute a volume of gas to said gas parameter detecting material.

The gas pathway may be configured to uniformly direct exposure of a volume of a user's inhalation and/or exhalation gases with said gas parameter detecting material.

The gas pathway may be configured to increase a gas parameter detecting material surface area in contact with a user's inhalation and/or exhalation gases.

The gas pathway may comprise a channel, and the cap may be configured to complete the gas pathway between the gas sampling portion and the indicator portion when attached to said body.

The gas pathway may be elongate.

The gas pathway may be an elongate channel or passageway.

The pathway may be spiralled.

The pathway may spiral radially outwardly from an outlet of said gas sampling portion, or an inlet into said indicator portion.

The pathway may provide for a gas path which is of a substantially greater distance than an internal radius of said indicator portion.

The gas pathway may provide for an indirect gas path, being of a substantially greater distance than a direct or straight-line distance between at least one port configured to vent to atmosphere from said indicator portion and an inlet to said indicator portion from said gas sampling portion.

The gas pathway may extend between at least one port configured to vent to atmosphere from said indicator portion and an inlet to said indicator portion from said gas sampling portion.

The gas pathway may provide for a substantially continuous path between an outlet of said gas sampling portion or an inlet to said indicator portion, and at least one port configured to vent to atmosphere from said indicator portion.

The at least one port configured to vent to atmosphere from said indicator portion may be located substantially adjacent a stopping flange portion of the body.

The stopping flange may be an enlarged region or portion of said body.

The stopping flange may be a region or portion of said body that is engageable with a portion of a device to which said breath indicator is attachable or engageable, to provide for a located position of said body relative to a said device or to provide for a maximum or a predetermined length (or depth) of insertion of said elongate body into a device.

The gas parameter detecting material may be one or more of: a gas detecting material, a gas composition detecting material, a gas humidity detecting material, a gas temperature detecting material, a carbon dioxide concentration detecting material or detector.

The gas parameter detecting material may be of a first colour and/or an initial visual state when a gas parameter is about the same or substantially similar to atmospheric air.

The gas parameter detecting material may be of a first colour and/or an initial visual state when exposed to gas containing or comprising up to about 0.04% or less carbon dioxide (atmospheric).

The gas parameter detecting material may be of a second colour and/or of a subsequent visual state when a gas parameter is greater than atmospheric air.

The gas parameter detecting material may be of a second colour and/or of a subsequent visual state when exposed to gas containing or comprising about 2% to about 3% or greater of a carbon dioxide concentration.

The gas parameter detecting material may be of a second colour and/or of a subsequent visual state when exposed to gas containing or comprising greater than about 0.04% of a carbon dioxide concentration, or greater than about 0.2% of a carbon dioxide concentration, or greater than about 2.5% of a carbon dioxide concentration.

The gas parameter detecting material may be of a second colour and/or of a subsequent visual state when exposed to gas containing greater a carbon dioxide concentration than atmospheric air.

A first colour and/or an initial visual state of said gas parameter detecting material may be one of: blue or purple.

A second colour and/or a subsequent visual state of said gas parameter detecting material may be one of: yellow or orange.

A change from a first colour and/or an initial visual state to a second colour and/or a subsequent visual state of said gas parameter detecting material may be based on a chemical reaction of said gas parameter detecting material.

The chemical reaction may be reversible.

A change from a first colour and/or initial visual state to a second colour and/or subsequent visual state of said gas parameter detecting material may be visually observable.

The indicium may provide for a greater contrast of colour or greater visibility with respect to said gas parameter detecting material to indicate either of a detected greater or lesser gas parameter relative to atmospheric air.

The indicium may provide for:

a first colour and/or an initial visual state with respect to said gas parameter detecting material when a gas parameter is the same or less than an atmospheric gas parameter, and/or

a second colour and/or a subsequent visual state with respect to said gas parameter detecting material when a gas parameter is the greater than an atmospheric gas parameter.

An initial and a subsequent indicium colour and/or visual states may be degrees of visibility.

A subsequent indicium colour and/or visual state may be of greater visibility or observability with respect to said gas parameter detecting material.

A subsequent indicium colour and/or visual state may be significantly more visible than an initial indicium colour and/or visual state.

There may be an initial indicium colour and/or initial visual state when said detecting material is a first colour and/or a first visual state.

There may be an initial indicium colour and/or an initial visual state when said gas parameter detecting material is exposed to gas containing or comprising up to about 0.03% or about 0.04% carbon dioxide (atmospheric).

There may be an initial indicium colour and/or an initial visual state when carbon dioxide concentration is substantially the same as atmospheric air.

A change from an initial indicium colour and/or an initial visual state to a subsequent indicium colour and/or a subsequent visual state may be based on a chemical reaction of said gas parameter detecting material.

A change from an initial indicium colour and/or an initial visual state to a subsequent indicium colour and/or subsequent visual state may be a visually or an optically observable change in contrast.

In a fifteenth aspect, the invention is said to broadly consist in a breath indicator for providing an indication of an inhalation and/or exhalation state of a user, said breath indicator comprising:

a body comprising a gas sampling portion configured for receipt of a user's inhalation and/or exhalation gases, and an indicator portion, said gas sampling portion in fluid communication with said indicator portion,

said indicator portion comprising a first region and a second region, one or both of the first and second regions comprising a gas parameter detecting material capable of changing between an initial visual state and a subsequent visual state indicative of detection of a gas parameter of inhalation and/or exhalation gases of said user;

wherein said initial visual state and/or said subsequent visual state of the first region visually contrasts with a visual state of at least said second region, and

wherein the body comprises a gas pathway between the gas sampling portion and the indicator portion, the gas pathway configured to expose a volume of gas to said detecting material.

In a sixteenth aspect, the invention is said to broadly consist in a breath indicator for providing an indication of an inhalation and/or exhalation state of a user, said breath indicator comprising:

a body comprising a gas sampling portion configured for receipt of a user's inhalation and/or exhalation gases, and an indicator portion, said gas sampling portion in fluid communication with said indicator portion;

said indicator portion comprising at least a first region and at least a second region, the first region comprising a gas parameter detecting material capable of changing from an initial visual state to a subsequent visual state indicative detection of a gas parameter of inhalation and/or exhalation gases of said user; and

wherein said initial visual state and/or said subsequent visual state of the first region visually contrasts with a visual state of at least said second region, and

wherein said indicator portion comprises a cap configured to substantially cover the detecting material and complete a gas pathway between a gas sampling portion and the indicator portion.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

The invention consists in the foregoing and also envisages constructions of which the following gives examples only.

It should be understood that alternative embodiments may comprise any or all combinations of two or more of the parts, elements or features or configurations as illustrated, described or referred to in this specification.

### BRIEF DESCRIPTION OF THE DRAWINGS

Specific configurations and modifications thereof will become apparent to those skilled in the art from the detailed description herein having reference to the figures that follow, of which:

FIG. 1 shows a representation of a typical system for resuscitating user or patient (such as an infant) as is known in the art, with a user or a patient receiving gases from a resuscitator that forms part of the system, the gases passing from the resuscitator to the user or patient via a manifold section, the manifold section connected to the resuscitator via a conduit that allows the gases to pass from the resuscitator to the manifold section.

FIG. 2 shows a cut-away view of the manifold section of FIG. 1 in more detail, the manifold section having a gases inlet which in use receives gases from the resuscitator, a gases outlet which in use passes the gases to a user or patient via a connected mask section (not shown), a device inlet which allows the passage of items such as suction tubes from the exterior to the interior of the manifold, and a PEEP outlet with a cap.

FIG. 3 shows a view of the manifold section of FIGS. 1 and 2 with a CO₂ breath indicator that forms part of the present invention located in the device inlet, the breath indicator being shown as hidden detail because it is inserted into the manifold.

FIG. 4A shows one arrangement of a breath indicator by itself, but which may be inserted into a manifold device, such as that shown in FIG. 3.

FIG. 4B shows another configuration of a breath indicator by itself, but which may be inserted into a manifold device, such as that shown in FIG. 3.

FIG. 5 shows a view of the manifold and breath indicator as shown in FIG. 4A with flow lines showing exemplar gases flow when the PEEP outlet is unoccluded, gases flow then the PEEP outlet is occluded and the movement of exhaled gases from a user or patient.

FIG. 6A shows a view of another configuration of a breath indicator with apertures in the breath indicator to allow gases flow through the breath indicator when the PEEP outlet is unoccluded.

FIG. 6B shows another configuration of a breath indicator by itself, but which may be inserted into a manifold device, such as that shown in FIG. 5.

FIGS. 7, 8 and 9 show alternative arrangements where the breath indicator may be attached to, or received by, a T-piece apparatus or a patient interface, such as a face mask, such an indicator may be that of the configurations illustrated by any of FIG. 4A, 4B, 6A, 6B,11-13 or 14-17.

FIG. 10A shows a further configuration of a breath indicator received by and attached to a T-piece, such a breath indicator as shown by FIGS. 11-13.

FIG. 10B shows a further configuration of a breath indicator received by and attached to a T-piece, such a breath indicator as shown by FIGS. 14-17.

FIGS. 11, 12 and 13 show in more detail a breath indicator, such a breath indicator of which can be attached to the T-piece of FIG. 10A. Such a breath indicator can be configured to be provided with a sensor or gas detector of the form in which an indicia may be provided directly or indirectly upon said gas detector or sensor or detector material, such as being for example any one of the indicia represented as in FIG. 19.

FIGS. 14, 15, 16, 17, 18 show in more detail a breath indicator, such a breath indicator of which can be attached to the T-piece of FIG. 10B.

FIGS. 14-16 illustrate a configuration in which an elongate body form is provided of a substantially tapered form. FIG. 15 specifically illustrates a further configuration in which a single or sole port or vent or outlet aperture is provided for discharge or venting of a sampled or sensed gas flow, as for example indicated by the arrow representing an indicative flow path of gas through said breath indicator. FIG. 17 illustrates a configuration in which a cap is shown in a disassembled or exploded view form from the remainder of the body of the breath indicator, while FIG. 18 illustrates this configuration in an assembled form.

FIG. 19 illustrates various representative indicia which may be used to help provide for a visual detection or visual change of state enhancement for a user with respect to a change in colour of gas detector or detecting material.

FIGS. 20A and 20B illustrate top and bottom perspective views of a further breath indicator embodiment comprising a directed gas path.

FIG. 21 illustrates a side view of the further breath indicator embodiment.

FIGS. 22A and 22B illustrate top and bottom views of the further breath indicator embodiment.

FIGS. 23A, 23B and 23C illustrate perspective cross sectional views and a side on cross sectional view of the further breath indicator embodiment, showing an example gas path.

FIG. 24A illustrates a complete view of the further breath indicator embodiment.

FIG. 24B illustrates the further breath indicator embodiment with a cap removed, showing a gas detector beneath.

FIGS. 24C and 24D illustrate exploded perspective views of the further breath indicator embodiment from above and below.

FIG. 25 illustrates a further configuration of the further breath indicator embodiment received by and attached to a T-piece.

### DETAILED DESCRIPTION OF SPECIFIC CONFIGURATIONS

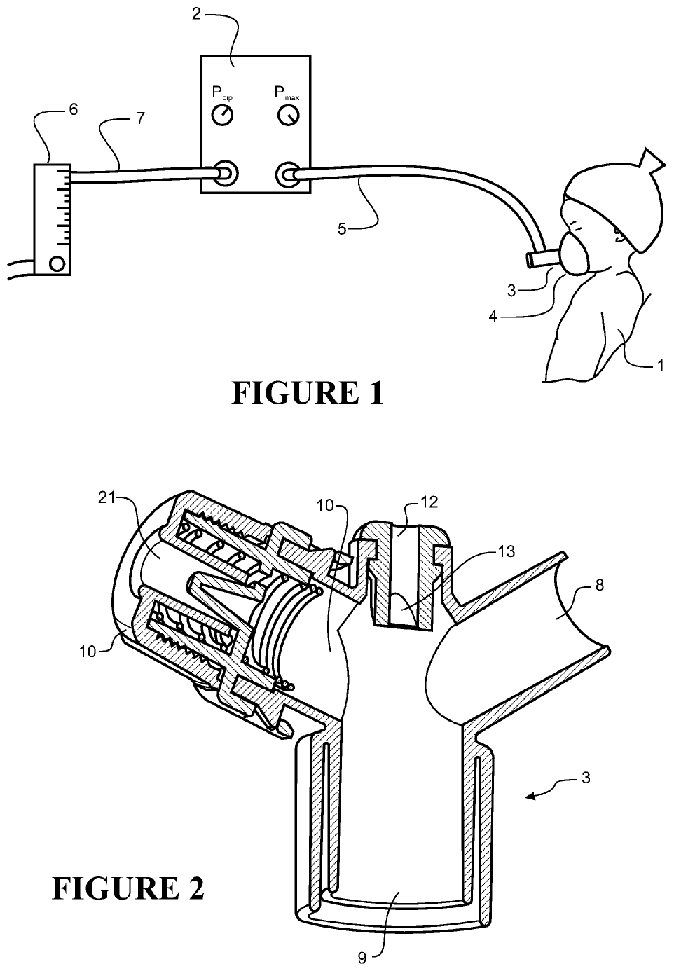

FIG. 1 shows a representation of a typical resuscitator system as is known in the art, with a neonatal infant 1 receiving gases from a resuscitator 2 that forms part of the system. A similar arrangement may be used for other users or patients if they are not an infant or neonate. The gases are passed on to the patient 1 via a manifold section 3 which is in use connected to a user or patient interface or mask section 4. The manifold section and resuscitation system may be used with a mask or an endotracheal (ET) tube. The configurations described below will be described with respect to a mask attached to the manifold, but it should be appreciated the use of such a breath indicator may be used in conjunction with ET tubes or other patient interfaces, such as face masks, nasal masks, oro-nasal masks, various configurations of nasal prong(s), and their associated connections or components.

It should be appreciated reference to a patient interface may comprise any one or combination of the following types: a face mask, an oral mask, an oro-nasal mask, a nasal mask, one or a pair of nasal prongs, an endotracheal tube, a T-piece resuscitator apparatus, gas flow regulator or gas pressure regulator associated with any one or more of these, although this list should not be seen as limiting.

The use of a mask with the resuscitation system is not to be considered limiting; it is simply an example as described by the following. The manifold section 3 is connected to the resuscitator 2 via a conduit 5 that allows the gases to pass from the resuscitator 2 to the manifold section 3. In the configuration shown, the resuscitator 2 is connected to a pressure regulator 6 via a gases supply conduit 7, the resuscitator 2 receiving gases from the pressure regulator 6 via the supply conduit 7, the pressure regulator fluidly (or gaseously) connected to a remote gases supply via a wall socket or similar. The pressure regulator 6 provides gases to the resuscitator 6 at 50 psi or thereabouts. In alternative configurations, the resuscitator could be a self-contained unit which draws in gases directly from atmosphere and passes these on to the user or patient 1 via the conduit 5. The gases or air supply are provided at a pressure above or at atmospheric pressure. The delivered pressure is varied between Peak Inspiratory Pressure (PIP) and Peak End Expiratory Pressure (PEEP) by the occlusion of a PEEP outlet (described later). The PIP is adjusted at the resuscitator 2 to a desired level. The system shown above may also be used for respiration and assisted respiration (ventilation) of a neonate or patient or another user or patient.

### Breathing Assistance Apparatus

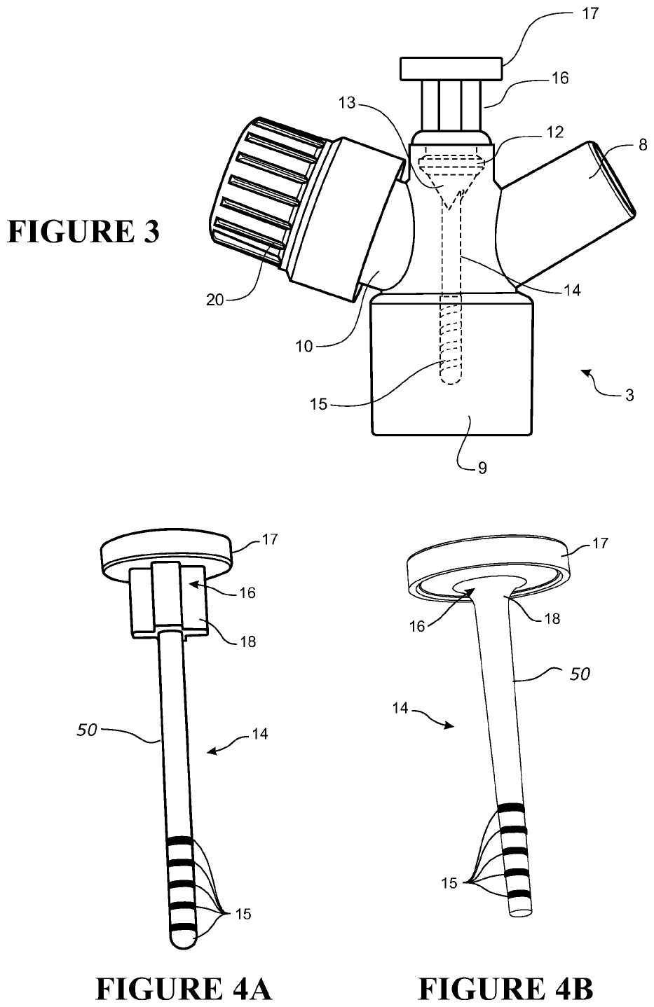

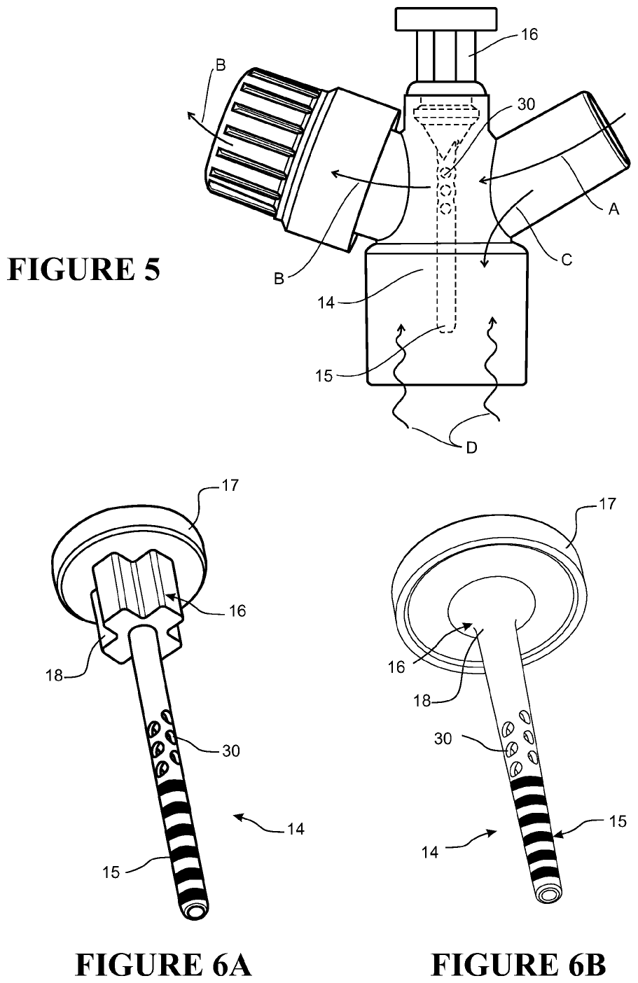

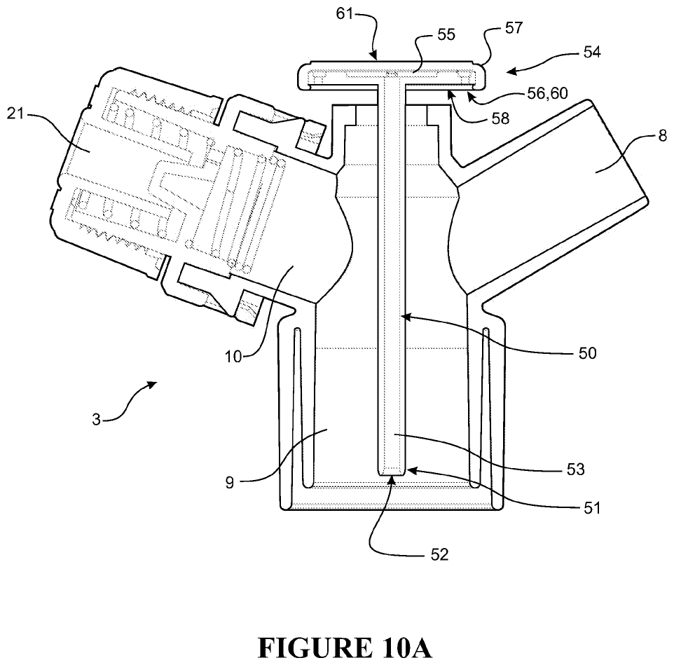

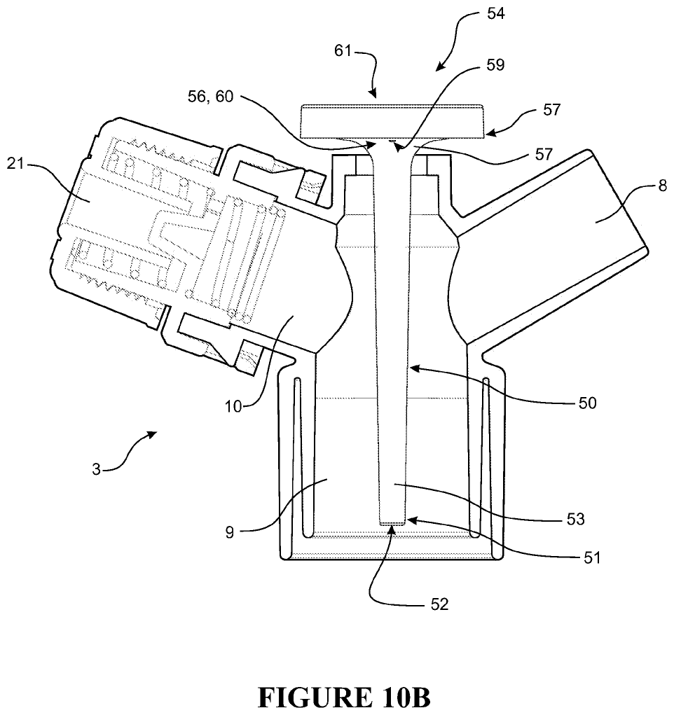

FIGS. 2 and 3 show one configuration form of the manifold 3 (and is further shown in FIGS. 5, 10). The main body of the manifold section 3 is substantially cylindrical in shape, with a plurality of inlet and outlet ports or port passages. The manifold section 3 is hollow and defines a gases space. The gases space forms gases connections between the inlet and outlet ports.

With reference to FIG. 2, manifold section 3 has a gases inlet 8 which in use is connected to the conduit 5 so that gases from the resuscitator 2 pass along the conduit 5 and are provided to the hollow interior of the manifold section 3. In use, the gases pass from the inlet port 8, through the hollow interior of the manifold section 3 and then exit the manifold section 3 via the gases outlet 9. In the configuration shown, the gases outlet 9 is connected to a detachable mask section 4 or similar (shown in FIGS. 1, 8, 9), the mask section 4 in use fitting around the nose or the nose and mouth of the user or patient 1, so that gases exiting the manifold section 3 via the gases outlet 9 pass to the user or patient 1 via the mask section. An example of a suitable mask section is disclosed in U.S. Pat. No. 574,487. All of the passages are defined by solid walls.

Also shown in FIG. 2 (with similar configuration also shown in FIG. 10A, 10B, 25) is a pressure relief section or over pressure relief section that forms part of the manifold section 3. In one configuration, a pressure relief section comprises a PEEP outlet passage 10 (PEEP outlet 10) passing outwards from the main body of the manifold section 3, with an aperture that opens to atmosphere located at or towards the outer end of the PEEP outlet 10. A cap 20 is positioned on the end of the passage 10 and may be used to adjust the valve mechanism. The cap 20 includes an aperture in it that allows gases to exit through the cap. The cap is moveable and can be moved to adjust the size of the aperture in order to control PEEP value.

The cap 20 includes an aperture 21 within the cap 20. The aperture 21 in the cap 20 can be occluded and unoccluded to control PEEP. In use a substantial portion, if not all of the gases from the inlet port 8, flow out of the second outlet passage 10 when the aperture 21 is open and uncovered. Only a small or no portion of the inlet gases reaches the PEEP outlet if the aperture 21 in the cap 20 is uncovered. In use, the aperture 21 in the cap 20 can be covered and closed or occluded to force the gases flowing into the inlet port to flow out of the PEEP outlet 9 and to the user or patient.

The aperture 21 can be opened and closed manually by a medical professional or user of the device using a finger or any other suitable body part or other suitable instrument. The delivered gases are varied between the PIP when the aperture 21 is occluded and the PEEP when the aperture 21 is unoccluded. The cap aperture 21 is opened and closed regularly to vary the PIP and PEEP at the normal rate of breathing to resuscitate a patient. The opening and closing of the cap aperture 21 provides gases to the user or patient in a cyclic manner. The opening and closing or occluding of the cap aperture 21 allows a user (such as medical professional) to simulate breathing of the user or patient for resuscitation or assisted breathing purposes. The manual occlusion of the cap aperture 21 also allows the user to control the amount and frequency of gases delivery to the user or patient.

In addition, a valve mechanism (not shown) may be positioned in association with the passage 10 and the aperture 21. The valve mechanism is adapted to control the flow of gases passing from the interior of the manifold section 3 to atmosphere so that the pressure in the manifold 3 will not rise above a certain pre-set level. If the pressure rises above such a level, the valve advantageously activates in such a manner that excess gases are vented from the manifold 3 and the pressure is limited.

Several types of valves are suitable for use as part of the pressure relief section, for example umbrella valves, jet valves, and so on. The purpose of the pressure relief section is to allow excess gases to be vented in the event of potentially harmful pressure build-up within the interior of the manifold section 3. In one configuration, there is no valve present in the second outlet passage 10. In an alternate form, the valve mechanism may be adapted to maintain the PEEP level reasonably constant. The valve may be similar to that described in U.S. Pat. No. 7,341,059.

A device inlet 12 is also shown as part of the manifold section 3. In one configuration, the device inlet 12 is located directly opposite the PEEP outlet 9. The device inlet 12 is intended to allow items such as suction tubes or similar to be inserted into the interior of the manifold section 3 during use, so that these items can then, if necessary, pass through the interior of the manifold section 3 to be inserted into the airways of the user or patient 1. The device inlet 12 can include a valve or similar which allows the passage of items from the exterior to the interior of the manifold section 3, but which seals to prevent the flow of gases from the interior to the exterior both during use when no items are inserted, and during the insertion of items. In one form, the device inlet 12 includes a duck billed valve 13.

The duck billed valve 13 is normally sealed, but upon insertion of an item e.g. a catheter, the duck-billed valve 13 opens to allow the catheter end to be received and enter the interior of the manifold section 3. The bill of the duck billed valve 13 seals around the end of the inserted item, thereby helping ensure that the manifold section 3 remains sealed against inadvertent gases leaks. Accordingly, in one configuration, the duck billed valve can be used to receive a breath indictor 14, 54.

### Breath Indicator

In various configurations, a breathing assistance apparatus may also have a separate breath indicator 14, 54, 204 which is a separate item to, and used in conjunction with, the manifold section 3 or with other devices, such as patient interfaces or other devices to be associated in close proximity to a patient's inhalation and exhalation gases. Such indicators 14, 54, 204 are removable from the breathing assistance apparatus. Such indicators may be provided separately or provided as part of a package with a breathing assistance apparatus, such as a patient interface or a resuscitator device or system.

In a general sense the breath indicator 14 comprises a body, having a sensing portion and an attachment portion 16. In one embodiment, the body is an elongate body 50. The sensing portion comprises a material or structures that switches between two visual indicator states.

The first visual indicator state relates to inhalation and the second visual indicator state relates to exhalation by the patient.

The detecting material or structures can change between the visual indicator states at a sufficient rate to substantially correspond to inhalation and exhalation changes of the patient to indicate inhalation and exhalation.

The indicator body may include another structure to reduce the indicator's resistance to flow.

The attachment end portion 16, 56 of the breath indicator 14 may include features or structures to allow the breath indicator 14 to be held in the breathing assistance apparatus, in use. The attachment end portion 16, 56 may also include structures to align the breath indicator's elongate body 50 in the correct orientation and position within the breathing assistance apparatus, or patient interface as necessary.

Referring now in particular to FIGS. 3, 4A, 4B, 5, 6A, 6B, the manifold section 3 is oriented so that the gases outlet 9 is at the lowest point, and the device inlet 12 is at the top of the device, directly above the gases outlet 9. This orientation generally corresponds with how the device will be oriented in use, with a user or patient 1 lying on their back facing upwards and the gases outlet 9 directly above their nose and mouth. The description below will assume this ‘in-use’ orientation for the purpose of describing the relationship of various features to one another. However, no other significance should be read into the use of terms such as, for example, ‘above’, ‘below’, ‘top’ and ‘bottom’ when used in this specification. These are merely intended to convey a relative relationship for a specific orientation—such as that which is shown in FIG. 4.

In one configuration, the elongate body 50 of the breath indicator 14 generally has the form of an elongate cylinder. The elongate body 50 has a sensing end 15 and an attachment end 16. One end of the elongate body 50 is a sensing end 15 and the other end is an attachment end 16. The sensing end 15 will be described in detail below.

In various forms, the elongate body 50 (or of any of the other embodiments) may be formed from a rigid plastic such as a polycarbonate or polypropylene.

In use, and for example as shown in FIG. 3, the sensing end 15 is inserted through the device inlet 12. The device may for example itself be or be connected to a patient interface, such as a full face mask, a nasal mask, an oro-nasal mask, a mouth mask, a cannula. The duck billed valve 13 seals around the elongate body 50 of the breath indicator 14 to maintain gases pressure integrity within the manifold section 3. The same configuration would apply in relation to breath indicator 54 when being inserted through the device inlet 12 and duck billed valve 13 (or a patient interface as listed above).

The breath indicator 14 or 54 or 204 may be about 30 mm to about 60 mm in length, or may be a length of about 50 mm. The length of the body of the breath indicator 14 or 54 or 204 may be greater than about 5 mm, or about 5 mm to about 50 mm, or about 40 mm to about 50 mm. The actual length will depend on the size of the device (such as a breathing apparatus or patient interface) to which the indicator 14 (or 54 or 204) is to be attached.

The indicator 14 may have an outer diameter of about 1 mm to about 5 mm, or may be about 3 mm. The actual diameter will depend on the size of the inlet of the device (such as a breathing apparatus or patient interface) through which the indicator 14 (or 54 or 204) is to be inserted.

The elongate body 50 may be of a reasonably smooth surface along its length. The small outer diameter of the elongate body 50 relative to the size of the gases space means the breath indicator 14 (or 54 or 204) does not need to occupy much room in the gases space. The shape of the elongate body 50 may assist in minimising resistance to flow of gases in the gases space.

The elongate body 50 may be provided with a taper or tapered form. The elongate body 50 may taper from a relatively smaller outer or external diameter substantially at or toward said gas sampling portion to a relatively larger outer or external diameter substantially at or toward said gas sensing portion.

When said elongate body 50 is provided with a taper, the taper may be from a relatively smaller outer or external diameter substantially at or toward said gas sampling portion to a relatively larger outer or external diameter substantially at or toward said gas sensing portion.

Where a substantially tapered configuration of the elongate body 50 is provided, the taper can extend from at or substantially toward the gas sampling portion toward the gas sensing portion about one quarter, or about one third, or about one half, two-thirds, three-quarters, or a whole of, the length of the elongate body 50.

In another configuration, the elongate body 50 may comprise a lead-in portion 51. Advantageously, such a lead-in portion 51 can be a region of the elongate body 50 being of a substantially narrowed (or more narrowed) external diameter. Alternatively, the lead-in portion 51 is a tip or end of said elongate body 50 comprising a chamfered edge or substantially radially inward curvature so as to assist with access or initial insertion of the body or elongate body 50 or gas sampling end into the device.

It should be noted that sensing end and attachment end of the indicator 14 are defined as the respective ends of the indicator's 14 elongate body 50. Sensing end 15 and attachment end 16 also encompass a distance of about 1 mm to about 15 mm from their respective ends. The meaning adopted is dependent on context. In the general sense when sensing end or attachment end is used in the specification, it is meant the ends of the elongate body or at least the general area of the elongate body around the ends, specifically in relation to the indicator 14.

### Sensing End

In use, the sensing end 15 of the indicator 14 comprises a detecting material that may change between two visual indicator states. Alternatively, the detecting material may comprise a visual indicator comprising a contrasting colour configured to contrast against an indicia as described herein,

The indicator 14, 54, 204 provides for a visual indicator and the detecting material can change between two visual states or between an initial visual state and a subsequent visual state. A visual indicator state is a physical visual state of the detecting material of the sensing end 15 or sensor 55 (not shown) for the indicator 14 or 54 or 204, respectively.

A visual indicator state is observable by a human eye and the difference between two visual indicator states is similarly observable.

The indicator 14, 54, 204 is adapted to detect inhalation and exhalation of a user or patient and visually signal inhalation, exhalation and the change between the inhalation and exhalation states. The detecting material changes between a first state or an initial state, relating to inhalation phase and a second state or subsequent, related to an exhalation phase. The detecting material is adapted to change states at a rate to substantially match inhalation and exhalation. The detecting material may be colorimetric, meaning the detecting material changes colour as it changes between the two states of inhalation and exhalation. Advantageously, the detecting of an inhalation and exhalation is to sense a gas parameter, such as the amount (or concentration) of CO₂ present. Other parameters may for example include, gas temperature or gas humidity or concentrations of other gases.

In one configuration, at least part of the sensing end 15 of breath indicator 14, or sensor 55 of breath indicator 54 or detector portion 230 of breath indicator 204, is adapted so that it will rapidly change colour in the presence of CO₂ concentrations above those normally found in atmospheric gases—i.e. it will change colour when it comes into contact with exhaled air from a person. The sensing end 15 or at least part of the sensing end of breath indicator 14, or sensor 55 of breath indicator 54, or detector portion 230 of breath indicator 204, is also formed and adapted so that it will change colour rapidly, if not almost instantaneously. Once the CO₂ concentration reduces back to, or close to, that of atmospheric air, the sensing end 15 or at least part of the sensing end will return to its original colour from the detection colour. In this manner the sensing end 15 of indicator 14, or sensor 55 of indicator 54, or detector portion 230 of breath indicator 204, is capable of changing from a resting or base or initial colour or visual state when exposed to atmospheric air, to a second or subsequent colour or detection colour or visual state when exposed to CO₂, and back again to the resting colour or initial visual state once the CO₂ concentration diminishes, and advantageously the colour change takes place rapidly enough to roughly match the inhalation/exhalation cycle of the user or patient 1. That is, rapidly enough to detect end-tidal or breath-by-breath Carbon Dioxide (CO₂) in the breath of a user or patient. Advantageously, the colour change takes place when the indicator dye or indicator material is exposed to CO₂ concentrations of 2.5% or above by gases volume. Likewise, the change may be detected and shown on accordance with a detected gas parameter, such as a gas temperature or a gas humidity or a concentration of another gas, relative to typical atmospheric gas conditions, or relative to measured inhaled gas conditions.

There are several ways in which the sensing end 15 of indicator 14, or sensor 55 of indicator 54, or detector portion 230 of breath indicator 204, could be formed.

As shown for example in FIGS. 6A, 6B, in one form of breath indicator 14, the sensing end 15 is formed as a hollow section which holds an indicator dye. The wall or shell of the hollow section has pores to allow gases, and especially exhaled gases with a high concentration of CO₂ to come into contact with the indicator dye. In use, the indicator dye changes colour when the CO₂ concentration rises above that normally found in atmospheric air, and a user can view this colour change through the wall of the manifold section 3 and the wall of the hollow section which holds the indicator dye.

In an alternative configuration, the sensing end 15 is formed by applying a layer of indicator dye, or a layer of material infused with an indicator dye, to the outside surface of at least the sensing end of the elongate body 50.

Alternatively, the indicator dye or indicator material may be applied to the elongate body 50 of indicator 14 in any other suitable way that allows CO₂ from the user or patient's exhaled air to come into contact with the breath indicator 14.

As outlined above, the indicator dye or indicator material changes colour when the CO₂ concentration rises above that normally found in atmospheric air, and a user can view this colour change through the wall of the manifold section 3 and if the dye is held in a hollow section, the wall of the hollow section which holds the indicator dye.

In a yet still further alternative, at least the lower end part of the elongate body 50 may itself be formed from a CO₂ detecting material, attached or connected in any suitable manner to the remainder of the breath indicator 14.

In relation to the configurations illustrated by FIGS. 4A, 4B, the sensing end 15 comprises a detecting or detector material which can be applied to the sensing end 15 in a manner so as to provide or leave portions of the sensing end 15 free of the detecting or detector material. Such free regions can have applied thereto an indicia to provide for a contrast state with respect to the colour changeable detector material, or alternatively, the colour changeable detector material may comprise the indicia to provide for a contrast state with respect to a background. Such a pattern or array of detecting material interspersed with an indicia may assist to provide an observer with an improved contrast state or condition to visually observe and detect a change in the colour of the detecting material. A similar arrangement is provided in relation to the illustrated configuration forms of FIGS. 6A, 6B (further details of which are provided below in relation to the provisioning of apertures 30).

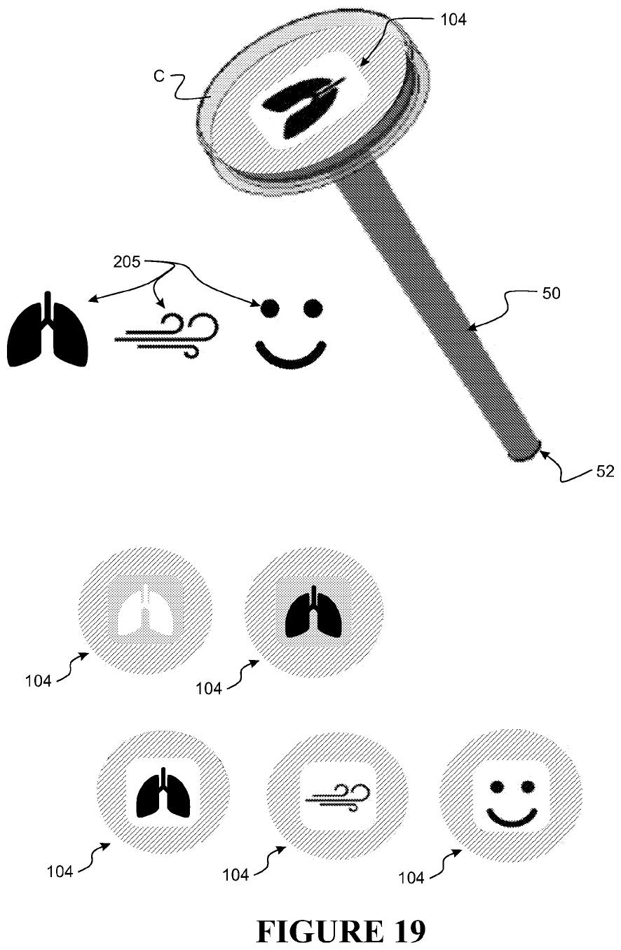

Advantageously, the detector material visually surrounds the indicia 104. Examples of icons or shaped indicia are shown as item 205 for example in FIG. 19.

The alternating arrangement of detector material and indicia is intended to provide for a system in which the ease of observing a change in colour of the detecting material is enhanced or emphasised for a person providing the visual detection.

Advantageously, the first or initial state is substantially the same or a similar colour or visual state to the detector material in ambient conditions.

It will be appreciated the colour or visual state of the indicia can be selected to enhance or emphasise or improve a contrast state or condition of the detecting material colouration between each of its colour or visual states or conditions between a targeted gas detection and a targeted non-gas detection (i.e. whether carbon dioxide is detected/sensed or not).

A further alternative configuration of the breath indicator 14 is shown in FIGS. 6A, 6B. As shown in FIGS. 6A, 6B, the apertures 30 are positioned or formed in the upper part of the breath indicator 14. The apertures 30 are in the part of the breath indicator 14 that is closest to the duck billed valve 13, meaning the aperture 30 are closer to the attachment end 16 than the sensing end 15. The apertures 30 may be about 1 mm to about 10 mm away from the duck billed valve 13. Alternatively, the apertures 30 may be closer to the sensing end 15, for example may be about 1 to about 10 mm away from the end of the breath indicator 14. As a further alternative, the apertures 30 may be positioned at any other positions or arrangements along the breath indicator 14, this form not being shown in the figures.

The apertures 30 of breath indicator 14 can be hexagonal in shape and arranged, for example, in a honeycomb shape. Alternatively, the apertures 30 may be any other suitable shape such as diamond shaped, rectangular, oval, circular etc. FIGS. 6A, 6B show the apertures 30 may be arranged in a honeycomb structure. The breath indicator 14 may be hollow with detector material inside the tube. Or, the detector material may be separately, or in addition, applied to the outside of the breath indicator 14 body near the sensing end 15.

When the cap aperture 21 of the device 3 is unoccluded, the gases received in the inlet port 8 pass through the apertures 30 and out of the cap aperture 21. This is shown by arrows A and B in FIG. 5. The gases from the gas supply (arrow A) are received by the inlet port 8 and pass through the apertures 30 and out through the cap (arrows B). When the cap aperture 21 is occluded the gases received by the inlet port 8 (arrow A) pass out through the gases outlet 9 and to the user or patient (as shown by arrow C). The gases exhausted out of the PEEP outlet 10 and cap aperture 21 cause the gases exhaled by the user or patient to be drawn upward and into the indicator through the apertures 30 (shown by arrows D). The gases exhaled by the user or patient enter the manifold 3 through the gases outlet 9 (arrows D), since the user or patient exhales into the mask 4 or ET tube (not shown) and the gases outlet 9. The exhaled gases are drawn upward due to a combination of venturi and Bernoulli or simply venturi or simply Bernoulli Effect. These effects may be caused by the pressurised gases (therapy gases) flowing from the inlet port 8 to the secondary outlet port 10 through the apertures 30. The pressurised gases flowing through the apertures 30 causes a pressure drop across the apertures 30 and from one side of the breath indicator 14 to the other. It is this pressure drop that can cause exhaled gases from the user or patient to be drawn toward the CO₂ detecting material. The gases exhaled by the user or patient are continuously drawn into the breath indicator 14 where they come into contact with the CO₂ detecting material and cause the CO₂ detecting material to change colour in the presence of CO₂ present in the exhaled gases of the user or patient. The draw created by the pressurised gases passing through the apertures 30 of breath indicator 14 may help to increase the sensitivity and response rate of the breath indicator 14. The system may be optimised based on the whole pattern design and by varying the size of the apertures 30. The apertures 30 can, as mentioned earlier, be hexagonal and arranged in a honeycomb pattern. Alternatively, the apertures 30 maybe any other suitable shape like square, rectangular, oval or circular.

The apertures 30 are advantageously positioned along the elongate body 50 of the breath indicator 14. At least some of the apertures 30 may be positioned such that they are aligned with the flow of gases from the inlet. The apertures 30 may help reduce the resistance to flow of gases created by at least part of the elongate body 50 being in the gases space 3. The apertures 30 being aligned with the flow of gases reduces the resistance to flow the flow of gases due to the apertures 30 and further reduces the resistance to flow of gases due to the presence of the breath indicator 14.

### Detector Material Composition

As outlined above, in one configuration, the breath indicator 14, 54, 204 comprises a CO₂ detector material in the form of a coloured dye or coloured material that changes colour in the presence of CO₂. The dye or material is capable of changing colour in the presence of CO₂ from the exhaled air of a user or patient. The change in colour corresponds to a particular visual indicator state. Advantageously, the detector material or dye changes colour when the user or patient's exhaled breath has at least an order of magnitude percentage change of CO₂ by gases volume. The indicator dye does not change colour in concentrations less than about 0.04% CO₂ hence the dye does not change colour due to CO₂ in the atmosphere. In one form, the composition of the detector material or dye is blue when it is not exposed to CO₂ or when the concentration of CO₂ is less than about 0.04%. The detector material or dye may gradually change from a blue to a yellow colour when it is exposed to CO₂ concentrations of about 0.2% or above. In an example embodiment, a visually contrasting change in colour may be observed when a change in CO₂ concentrations to between about 2% and 3%, or above about 2.5%. Alternatively, any other colour change is acceptable. A blue to yellow colour change may be utilised since it is very visible to a medical professional that may be monitoring the breathing of the user or patient. The blue to yellow colour change provides a visual indication to an observer of the user or patient's breathing. The detector material or dye changes colour due to a chemical reaction between the detector material or dye and the CO₂. The reaction is reversible and the detector material or dye changes colour back to blue when the CO₂ concentration drops an order of magnitude change, for example about 0.2%, or about 0.04%. The reversible reaction allows for a breath-by-breath indication, meaning each time the user or patient exhales the dye changes from blue to yellow and each time the user or patient ceases exhaling, and the indicator's sensing end 15 of breath indicator 14, or sensor 55 of breath indicator 54, or detector portion 230 of breath indicator 204, is exposed to atmospheric gases, the detector material or dye changes from yellow to blue. Breath-by-breath indication is advantageous because it allows a medical professional to monitor the user or patient breathing and ensure the user or patient is breathing correctly. The blue colour of the detecting material represents the first visual indicator state. The blue colour (first visual indicator state) corresponds to the inhalation of a user or patient. The yellow colour of the detecting material represents to the second visual indicator state. The yellow colour (second visual indicator state) corresponds to exhalation from the user or patient. As noted previously, other gas parameters may also be detected and responses provided by the detector material, for example in response to a detected gas temperature, or gas humidity, or a different gas concentration.

The detecting material may undergo intermediate visual indicator state changes. The intermediate states occur as the detecting material changes between the first and second visual indicator state. There may be two intermediate visual indicator states. Alternatively, there may be any other number of intermediate visual indicator states. The detecting material may go through a green phase and yellow green phase as it changes colour from blue to yellow. The green phase occurs when the CO₂ concentration is approximately between about 0.2% and about 2%. A yellow green coloured state occurs when the concentration of CO₂ is between approximately about 1.2% to about 2% or up to about 2.5%, and a colour change (or change in visual state) continues to occur as the detected concentration of CO₂ increases. The detecting material (indicator) changes colour to a yellow colour when the concentration of CO₂ is above approximately 2.5%. The concentration of CO₂ in the room air is generally around 0.03% to about 0.04%. Advantageously, the detecting material remains permanently yellow coloured if it is damaged or malfunctioning. This allows a user or a medical professional or supervisor of therapy to see if the indicator is damaged or malfunctioning. The breath indicator 14, 54, 204 can be changed. In one configuration, an optimal time of use of the breath indicator 14, 54, 204 is up to 24 hours, but this will depend on how much use the detecting material can take before it is exhausted. If the indicator is used for longer than 24 hours, the colour of the detecting material eventually fades to a yellow-light green colour—this may be useful as a further visual indicator to the user for indicating when the sensor or detecting material is nearing its end of life or exhaustion point. Between a first colour or a first visual state or an initial colour or an initial visual state, and a second colour or a second visual state or a subsequent colour or a subsequent visual state, there may be intermediate colours or intermediate visual states provided for.

The detecting material or indicator dye can be formed from any suitable dye such as, for example, metacresol purple or bromothymol blue. Any other suitable dye may also be used as an alternative. The dye must be a colour that is easily visible to the human eye when activated. The number of visual indicator states that may occur are dependent on the type of dye or material used as the detecting material. The breath indicator 14 visually represents inhalation and exhalation of a user or patient, with a clear visual state change between the inhalation phase and exhalation phase of the user or patient. The purpose of the breath indicator 14 is to visually show or signal inhalation and exhalation of a user or patient being resuscitated or ventilated to allow a medical professional to determine if a user or patient is breathing correctly or if the user or patient has been resuscitated.

In alternative forms the breath indicator's sensor end 15 or sensor 55 may be formed from, or may include, any other suitable substance to act as a CO₂ detector. In one form the sensing end 15 or sensor 55 of the breath indicator 14 or 54 or 204 respectively, may be formed or comprise a powder or grains of pH sensitive material. In other forms, the sensing end 15 or sensor 55 of the breath indicator 14, 54, 204 respectively, may be in the form of pH sensitive paper, such as litmus paper. Alternatively, the breath indicator's detecting material substance may be any other pH sensitive material that is known to persons skilled in the art, for example, phenol red, cresol blue, phenolphthalein, thymol blue, bromthymol blue and so on. In a further alternative form, the breath indicator's detecting material with sensing end 15 or sensor 55, or detecting material 230, for breath indicators 14 or 54 or 204 respectively, or may include substances that absorb and react with gaseous carbon dioxide to produce a colour change. Examples of such materials include, but are not limited to, are barium oxide, lithium hydroxide, calcium oxide, sodium hydroxide and many others that are known in the art. These CO₂ absorbing materials may be in any suitable form for example a lattice, powder, liquid or any other suitable form.

In another alternative configuration, the breath indicator 14 or 54 or 204 may comprise temperature sensitive materials that change colour in response to a specified temperature change. The purpose of the breath indicator 14, 54, 204 is to show breathing of a user or patient by indicating when a user or patient exhales. Exhaled air is generally at a higher temperature than ambient air. The temperature sensitive material changes colour when there is a predetermined temperature change, hence changing colour when a user or patient exhales. The temperature sensitive material may be arranged in any suitable form for example a strip, a disc, a powder and so on.

In a further alternative configuration, the breath indicator 14 or 54 or 204 may comprise a humidity sensor. Medical gases or medical air and oxygen used to resuscitate a user or patient are generally dry. In most cases, the gases or air or oxygen is humidified to less than 10% moisture content. The exhaled breath from the user or patient is substantially higher in terms of relative humidity and moisture content. The higher moisture or humidity of the exhaled air causes the indicator to change colour. The humidity sensor may only changes colour when there is a predetermined humidity change or when a predetermined level of humidity is reached. The humidity sensor is arranged in any suitable form for example a strip, a disc, a powder, as a rod and so on. The humidity sensor may also be a holographic sensor that may display a plurality of images. One image may correspond to low humidity and hence correspond to the inspiration phase of the user or patient breathing. The sensor image may change when there is predetermined change in humidity or when a predetermined level of humidity is reached, signifying exhalation or expiration phase of the user or patient.

In another alternative form, at least part of the inner surfaces of the manifold 3 may be coated with a CO₂ indicating material. The CO₂ detecting material may be applied or be present on the inner surfaces of the outlet 9. The CO₂ indicator may be present on all the inner surfaces of the gases outlet 9 or may only be present on parts of the inner surfaces of the gases outlet 9. Alternatively, or in addition, the CO₂ material may be present on the inner surfaces of other parts of the manifold 3 for example on the inner surface of the wall that defines the secondary outlet passage 10. In such a configuration, the indicia can be provided directly upon the detector material or may be provided in a similar arrangement or pattern or sequenced array such as that illustrated in FIGS. 4A, 4B. The indicia may also be any one or more of those for example illustrated in FIG. 19 to provide for an emphasised contrast state or condition with respect to the change in colour of the detecting material.

### Attachment End

The attachment end 16 of breath indicator 14, or the attachment end 56 of breath indicator 54 or 204, is formed as a handle to allow a doctor or other medical professional to insert or remove the breath indicator 14, 54, 204 from the manifold section 3. The handle may be formed so that a handle flange 17, 57 extends outwards perpendicularly from the top of the attachment end 16, 56, the handle flange 17, 57 allowing a doctor or user to gain a grip on the attachment end 16, 56 to pull the breath indicator 14, 54, 204 clear of the manifold section 3. In one option, the part of the body directly below the handle flange 17, 57 can be formed as a knurled or ridged grip for the same reason—to allow a person to grip the handle section more easily.

In some configurations, a stopping flange 18, 58 can be located at a mid-point of the elongate body 50 of the breath indicator 14, 54, 204 just below the knurled portion of the attachment end 16, 56. The stopping flange 18, 58 may extend outwards perpendicularly from the elongate body 50. In use, the sensing end 15 or sampling end 51 is inserted into the device inlet 12 and the elongate body 50 of the breath indicator 14, 54 is pushed through the device inlet 12 until the stopping flange 18, 58 contacts the top surface of the manifold section 3 and is prevented from travelling any further relative to the manifold section 3. Mid-point in this context is not intended to a halfway point, merely a point at some location between the two ends of the body or elongate body.

As illustrated by FIG. 4B, 6B or any of 10B, 15, 16, 18, a stopping flange 18, 58 may be provided in the form of a curved surface which transitions from a sensing end flange 17, 57 tapering inwardly (i.e. becoming more narrow or narrower) toward the elongate body 50. Such a curved transition provides a region which is capable of engaging with a device into which said breath indicator is to be inserted, and may provide for a gradual increased sealing or stopping force function due to the curvature of the region. Such a stopping flange may be the region of the elongate body 50 expanding radially outward as it joins to a gas sensing end of said breath indicator 14.

### Locking Feature

In one form, the breath indicator 14, 54, 204 fits into the device inlet 12 by means of a friction fit with the sides of the device inlet 12.

In alternative forms, either the breath indicator 14, 54, 204 or the manifold section 3, or both, can include an optional locking feature that locks the breath indicator 14 in place when the breath indicator 14 is inserted into the manifold section 3. The breath indicator 14 may be attached in place by any suitable connection or locking feature, such as a threaded connection or interference click-fit connection. The locking feature may be a protrusion or a series of protrusions (not illustrated) extending from the elongate body 50 just below the stopping flange 18, 58. The manifold section 3 itself may include at least one corresponding fastening feature to correspond with the locking feature on the breath indicator 14, 54, 204. Such a fastening feature can be positioned on, along, within or around the inlet 12. The mutually adapted fastening features could be, for example, at least one or a plurality of vertically aligned slots (not illustrated) positioned around the device inlet 12, which correspond with at least one or a plurality of ridges on the body of the breath indicator 14, 54, 204.

In another form, the breath indicator 14 can comprise an alignment feature or features (not shown). It should be noted that there may be one or multiple alignment features provided with the breath indicator 14. The alignment feature may form part of the locking feature/features. Alternatively, the alignment feature may be separate to the locking features. The alignment feature acts to provide correct positioning of the breath indicator 14 within the manifold such that at least some of the apertures 30 of indicator 14 are substantially aligned with the gases flow into the manifold 3. The apertures 30 being aligned with the gases flow are advantageous because there is no increased resistance to the flow of gases. The apertures being aligned with the flow may reduce the resistance to flow of gases from the inlet. The apertures 30 allow gases to flow through and hence reduce the resistance to flow due to the indicator being inserted into the gases flow. At least part of the gases flow through the apertures 30 rather than just around the indicator hence reducing the resistance to flow due to the indicator being inserted in a gases flow. The apertures being aligned with the flow of gases further goes in some way to reducing the resistance to flow due to the apertures 20. The apertures 30 being aligned with the flow allow the gases to flow more easily through the apertures and encounter less resistance to flow and fewer obstacles and can adopt a less tortuous path to flow around and/or through the indicator. The apertures 30 being aligned with the flow of gases reduce the resistance to flow of gases from the inlet.

The alignment feature may be any suitable feature. Some examples of an alignment feature are a baffle, a flange, a pin, a screw or any other suitable feature. The alignment feature can be positioned at or near the attachment end 16 of breath indicator 14. The alignment feature is advantageous because it allows the breath indicator 14 to be inserted and retained in the correct operational position. The alignment feature also makes using the breath indicator 14 easy for a user, because the alignment feature acts a guide for the correct position of the breath indicator 14. The breath indicator 14 may also comprise a receiving feature (not shown). The receiving feature may receive the alignment feature to correctly position the breath indicator 14, particularly the apertures 30 such that the apertures 30 are substantially aligned with the gases flow into and through the manifold 3.

Alignment features may also be provided on each of the breath indicators 54, 204.

### In Use

As outlined above, the breath indicator 14, 54, 204 is, in-use, inserted through the duck billed valve 13 and the sensing end 15 of breath indicator 14, and sampling end 51 of indicator 54 or 204, extends into the gases space defined by the manifold section 3, or into the space encapsulated or sealed (or unsealed) space of a patient interface. In use, the breath indicator 14 and particularly its sensing end 15, or sampling end 51 of breath indicator 54 or 204, is provided at least proximate to the mouth and nose of the user or patient 1, or region where gas is exhaled by the patient.

The sensing end 15 of breath indicator 14 can be about 1 mm to about 15 mm away from the nose and mouth of the patient. It should be understood proximate is about 1 mm to about 20 mm, but may be about 1 mm to about 10 mm away.

The sensing end 15 of the breath indicator 14 can be located proximate to at least the outlet 9. The breath indicator 14 can extends into the manifold section 3 until the sensing end 15 is between 2 mm and 8 mm away from the gases outlet 9. That is, between 2 mm and 8 mm from extending out beyond the gases outlet 9. The sensing end 15 can be approximately 3 mm away from the gases outlet 9. The extent to which the breath indicator 14 is located or inserted into the manifold section 3 may be provided such that the sensing end 15 can receive a suitable gas sample without contacting a patient.

In use, the user or patient 1 inhales gases passing out of the gases outlet 9 in a constant stream. The user or patient 1 will exhale against this constant stream. However, a portion will return into the manifold section 3 against the constant flow of gases. As the sensing end 15 of the breath indicator 14, or sampling end 51 of breath indicator 54, is positioned as close as possible to the gases outlet 9, there will be a greater concentration of gases at this point than there would be if the sensing end portion 15 or sampling end 51 were located at a point further away from the user's mouth or airways. The sensing end 15 or sampling end 51 is located as close as possible to the user's mouth and nose as possible without actively interfering with therapy.

It can therefore be seen that the sensing end 15, or sampling end 51, being extended into the manifold or space provided by a patient interface is advantageous because it allows the sensing end 15 or sampling end 51 to be at the point of most sensitivity and therefore provide an optimised measurement because it is as close as practicable to the patient. The location, or locating, of the sensing end 15 or sampling end 51, as described above, is also advantageous because there is preferentially no increase in dead space within the manifold 3 involved in making the measurement, or of a patient interface configuration.

If the breath indicator 14, 54, 204 and more specifically the sensing end 15 or sampling end 51 is located in its own sub-passage or recess, this can form a deadspace where CO₂ concentration may not be an accurate reflection of that exhaled (or inhaled) by the user or patient 1.

The elongate breath indicator 14, 54, 204 is also advantageous because it can be inserted through the duckbilled valve 13, firstly allowing the therapy to continue as the breath indicator 14 is inserted in or removed from the manifold 3, and secondly because the indicator sensing end 15 or sampling end 51 can be pushed to a point where it is closest to the exhaled gases from the user or patient 1, therefore providing or sampling gas exhaled by the patient for providing an as accurate a reading as possible.

The volume of air exhaled by an infant or neonate (tidal volume) is generally very small, e.g. generally in the range of 1 to 40 millilitres. The volume is typically larger for adults. Accordingly, the breath indicator 14 may be capable of measuring and detecting CO₂ in such small tidal volumes. The sensing end 15 or sensor 55 of breath indicators 14, 54, 204 is advantageous because it can measure small tidal volumes. Further, providing the breath indicator 14, 54, 204 with sensing end 15 or sampling end 51 to extend close to the patient allows the breath indicator 14, 54, 204 to measure or sense from sampled gases small tidal volumes from the patient.

As part of an exhalation of gases from a patient, the exhaled gas stream may travel back into the manifold 3. The exhaled gases become diluted as they travel through the manifold 3. The exhaled gases are diluted by the therapy gases received by the manifold 3 through the inlet port 8. The sensing end 15 or sampling end 51 may be about 2 mm to about 8 mm away from the gases outlet allows the sensing end 15 to be exposed to the highest concentration of exhaled gases, or in the case of sampling end 51 directing the gas to sensor 55 of breath indicator 54 or 204, that is before the exhaled gases become too diluted by the therapy gases. This is advantageous because it allows for an accurate representation of a user or patient's breathing. The CO₂ detecting material is adapted to detect very low tidal volumes of CO₂. This makes the breath indicator 14, 54, 204 advantageous since the breath indicator 14, 54, 204 can detect very low tidal volumes.

The breath indicator 14 shown in FIGS. 5 and 6A, 6B is advantageous because the exhaled gases are drawn into elongate body 50 due to either the venturi effect or Bernoulli Effect. This is advantageous because the draw allows a substantial part of the exhaled gases to be passed over the CO₂ detecting material. This draw of exhaled gases allows the breath indicator to be more accurate since a substantial part of the exhaled gases passes over the CO₂ detecting material without getting diluted by the incoming therapy gases. In this manner, an enhanced contrast state or condition can be provided by the additional inclusion of the indicia.

The breath indicator 14 being an elongate shape, and optionally being of a tapered form substantially as described above, is advantageous because it does not increase the resistance to the flow of gases in the manifold 3. The breath indicator 14 with elongate body 50 also reduces the resistance to the flow of gases from the inlet port 8 to the gases outlet 9. The outer surfaces which are exposed to gases flow may be smooth to not substantially increase the resistance to gases flow. The apertures 30 in the breath indicator 14 as shown in FIG. 6 also contribute to reducing resistance to flow. The apertures 30 allow gases to flow through the breath indicator 14 as gases flow from the inlet port 8 to the PEEP outlet 10. In addition to this the breath indicator 14 being an elongate shape as shown in FIG. 3 reduces the dead space of the gas manifold 3 increasing the sensitivity of CO₂ measurement detection.

In a further form, the breath indicator 14, 54, 204 and breathing apparatus 3 as described above may be sold or packaged or be in the arrangement of a kit of parts. The kit may be in any suitable form.

Instructions of assembling, using and maintaining the breath indicator 14, 54, 204 and breathing apparatus 3 will be supplied with the kit. In use, the breath indicator 14, 54 is inserted into the breathing assistance apparatus 3 through a device inlet 12, the device inlet 12 including a duck billed valve 13, or inserted into a patient interface. The breath indicator 14, 54, 204 can be locked in place by an optional locking feature. However, the indicator 14, 54, 204 can be held in place by friction forces with a duck billed valve 13, or other port of a patient interface (such as a mask) or T-piece.

### Further Configuration

As discussed above, features of use, detector material or sensor may be applicable to the configuration described in more detail below.

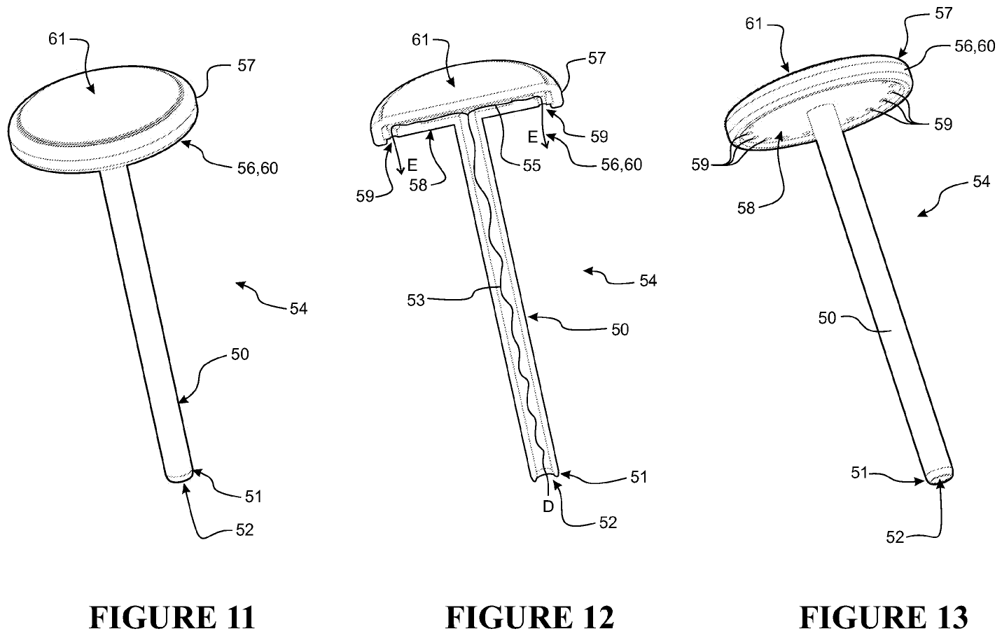

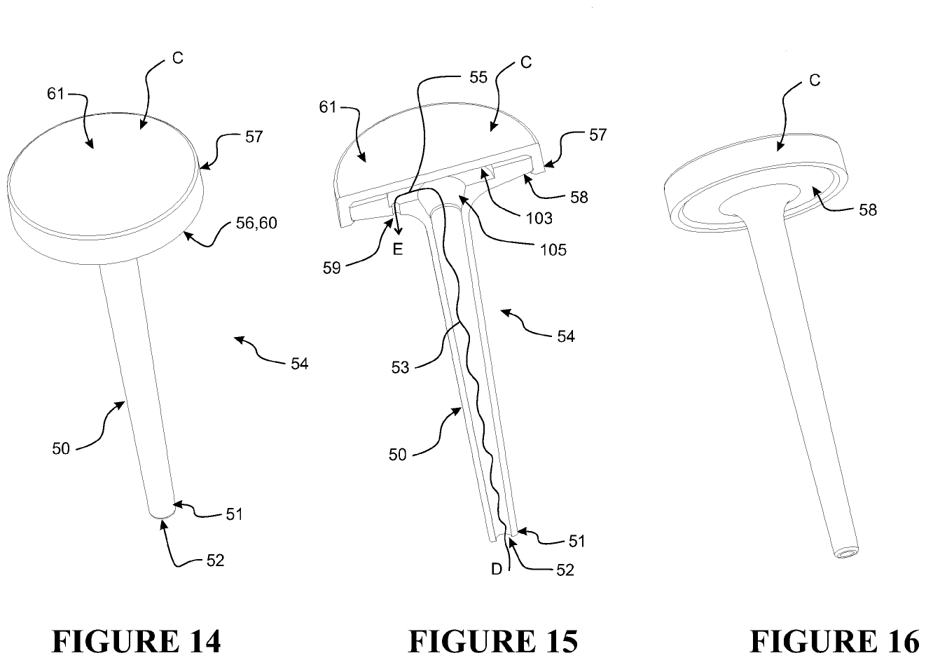

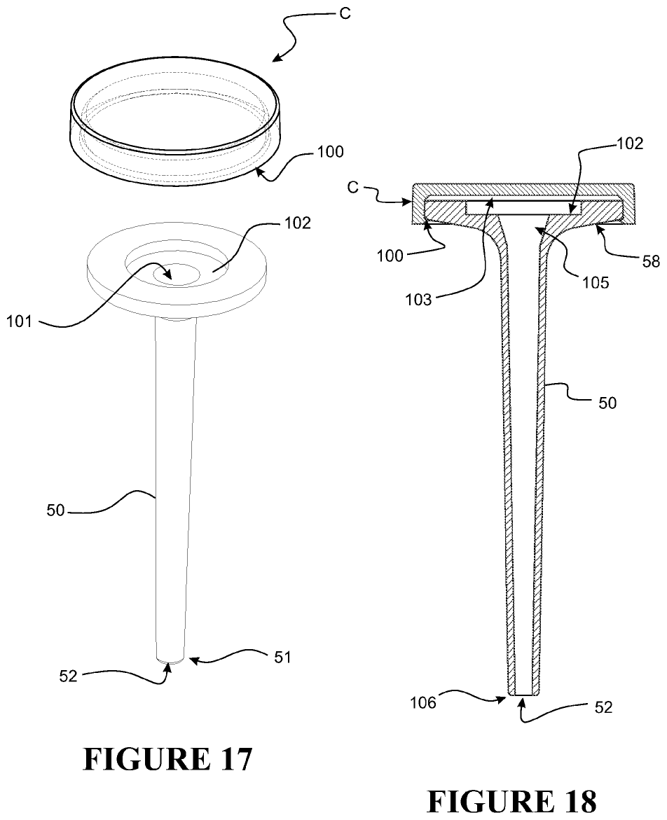

In a further configuration as illustrated in FIGS. 11 to 19, there is provided a breath indicator 54 that is receivable by a part of a breathing assistance apparatus (or patient interface), such a breathing apparatus capable of supplying gas to a patient. It will be appreciated a breathing apparatus may include a ventilator system or resuscitator system, or parts of these, such as T-piece 3.

Such a breath indicator 54, 204 comprises an elongate body 50 having a gas sampling end 51, and an attachment end 56. The attachment end 56 adapted to attach to a part of a breathing assistance apparatus (e.g. via a duck-billed valve of a T-piece) and for locating the gas sampling end 51. The gas sampling end 51 is locatable in a region where gas from the patient is to be exhaled. The gas sampling end 51 may be in communication with a sensor (not shown, but to be located at 55) comprising a detector material. The detector material is changeable between a first or initial visual indicator state (relating to an inhalation phase of the patient), and a second or subsequent visual indicator state (relating to an exhalation phase of the patient). The detector material also being is capable of changing between the visual indicator states at a sufficient rate to substantially correspond with the inhalation and exhalation phases of the patient connected to the breathing assistance apparatus.

As discussed generally in relation to the previous configurations, the sensor comprises gas detecting material. Examples of gases that may be detected include nitrogen, oxygen, carbon dioxide, argon or any component of air. The detecting material used is that which can change from a first visual indicator state (e.g. first or base colour) to a second indicator state (e.g. second or indicator colour) when exposed to gas having CO₂ concentration greater than that normally found in atmospheric air. Similarly, the detecting material can change from the second visual indicator state to the first indicator state when exposed to gas having CO₂ concentration the same or substantially similar to those normally found in atmospheric air. The material chosen is advantageously that which changes from the first visual indicator state to the second visual indicator state when exposed to gas having a concentration by volume of at least about 2.5% or more CO₂.

Advantageously, in this configuration of the breath indicator 54 or 204, the visual indicator state of the detecting material can be optically or visually observed by a user of a breathing assistance apparatus (e.g. ventilator or resuscitator system) for a patient. One other particular use of such a breath indicator is the ability for a user to detect the end-tidal or breath-by-breath CO₂ present within gas being exhaled by the patient. Such detection is enabled by the user optically or visually observing the change in visual indictor state of the detecting material.

In various configurations, at least a part of the attachment end 56 of the breath indicator remains external of a part of a breathing assistance apparatus (e.g. patient interface such as a T-piece or mask) to which the breath indicator 54, 204 is attached.

The attachment end 56 can be provided in a substantially perpendicular orientation to the elongate body, and may for example be substantially disk-shaped, such as that shown in FIGS. 10A, 10B, 11-13, 14-16, 17, 18.

It should be further appreciated that the attachment end 56 can be formed to be, or so used as, a handle portion including a handle flange extending outwards perpendicularly from the end of the attachment end 56. This may enable a user to better grip or hold the breath indicator 54, 204 when removing the indicator 54, 204 from a patient interface or when inserting the indicator 54, 204 into an interface (or part of a breathing assistance apparatus).

Further, the breath indicator 54, 204 may further comprise a stopping flange 57 (not shown, but could be similar to that of FIGS. 3-6) that is located at least partway along the elongate body 50 and which extends substantially or generally perpendicularly from the elongate body 50. Such a flange 57 can be located between the attachment end 56 and the sampling end 51, and may be a region of curved surface providing for a wider neck closer to the sensing end and a narrower neck closer toward the gas sampling end 51.

In a similar manner to the configurations previously discussed, the exterior surface of the elongate body 50 may be substantially smooth, or otherwise shaped, such that, in-use the elongate body 50 does not increase the resistance to flow of gas through the breathing assistance apparatus to which the indicator 54, 204 is attached. As with earlier described configurations, the elongate body may be of a tapered form (the earlier configurations of which are referred to herein).

The elongate body 50 is provided such that it has a length sufficient to allow the gas sampling end 51 to be located in a region where gas from the patient is to be exhaled, and where the attachment end 56 is located in a position attached to a part of a breathing assistance apparatus.

In one example, the elongate body 50 may have an outer diameter of about 1 mm to about 5 mm, and a length of about 30 mm to about 60 mm. It will be appreciated different lengths or elongate body 50 diameter may be used depending on the application and size of breathing apparatus or interface (or patient).

The gas sampling end 51 of the elongate body 50 comprises a gas inlet 52, the inlet 52 being provided for fluid communication with the sensor via a passageway 53. The sensor comprising the detecting material is advantageously provided in-line with the passageway 53, such that the gas flowing through the passageway 53 contacts the sensor (with detecting material) for providing the visual indication of change in gas components (e.g. with respect to CO₂). Accordingly, the passageway 53 is provided for extending from the gas sampling end 51 of the elongate body 50 to the sensor.

In more detail, the passageway 53 provides for a gas flow path D, 240 extending from the gas inlet 52 to one or more ports 59. Such port 59 are provided to be in fluid communication with the surrounding atmosphere external of the resuscitation system. That is, the ports 59 are provided for allowing the venting of gas flowing through the passageway 53 to the surrounding environment outside of the breathing apparatus, such as flow path E, 240. In some configurations, a single port 59 may be provided.

It should be appreciated by one of skill on the art that a benefit of providing a gas flow path D, 240 to ambient or atmospheric conditions allows for continual flushing of the gases through the breath indicator, causing a faster response in the detector material over an in-line system (such as those available in the prior art). The faster response allows for a greater change in the colour of the detector material on a breath-by-breath basis, which is more readily observed by the carer. Moreover, this observation of colour-change (indicating breathing) is additionally enhanced by use of an indicia, as described elsewhere herein.

In some configurations, the port 59 or one or more ports 59 (if provided) may be sized so as to accommodate for a maximum flow rate or vent rate or gas flow rate (to provide for control of the gas being vented or discharged to the surrounding environment from within the breath indicator 54).

Therefore, the sensor is positionable for contact with the gas flow path D, 240 through the passageway 53 is in contact with gases exhaled by a patient when, in a first mode of use such gases flow in through the gas inlet 52 of the sampling end 51, through the passageway 53 and make contact with the sensor. In this mode, a change in the sensor's detector material from the first visual indicator state to the second indicator state (if the CO₂ concentration is sufficient) may be enabled. And, in a second mode of use, such as when a patient is in an inhalation phase, gas flow passing through the patient interface enters the gas inlet 52 at the sampling end 51, flows through the passageway 53, and contacts the sensor. In such a second mode, the flow of gases being detected by the detecting material allows the change from the second visual indicator state to the first visual indicator state. Such a second mode flushes gases provided to the patient interface or breathing assistance apparatus through the breath indicator 54.

In one configuration, the sensor with detecting material is located in a region adjacent the attachment end 56. In this setup, the attachment end 56, or at least a part of the attachment end 56, is formed of a visually transparent material allowing a user to visually detect changes between the first and second visual indicator states of the detector material (i.e. between the two modes of use and for monitoring the inhalation and exhalation phases of a patient). For example, the top surface 61 of the attachment end 56 may be suitable optical transparency.

In this respect, a detecting material capable of changing colour from a first colour to a second colour according to inhalation and exhalation is provided, and a component comprising at least one indicia changeable as to contrast or visibility according to a change in colour or response of said detecting material with said user's inhalation and exhalation gases is provided. That is, an indicia may be provided either directly upon or adjacent to said detecting material or may be provided indirectly thereon by provisioning of a separate component. In this manner, a visual contrast state or condition may be improved or emphasised when the detecting material changes colour and an observer is able to visually detect with improved ease the change in colour of the detecting material.

Accordingly, the attachment end 56 is enabled with a housing or hollow section 60 for holding or retaining the sensor in position, such that the sensor is in contact with the gas flow path of gas flowing through the passageway 53 prior to being expelled to the external environment out of the ports 59. The housing 60 or attachment end 56 (or parts of each) may be of visually transparent materials enabling a user to observe the changes between the visual indicator states of the detecting material. The housing 60 can be provided by a cap C, 61 which includes a radially inward projection or ledge or other retaining surface feature for engaging with a perimeter or circumferential portion of the body of the breath indicator 54.

As discussed previously, the breath indicator 54 as described above may be utilised together with a T-piece, or patient interface. In such an arrangement, the manifold of the T-piece, when in function, has higher pressure than the atmosphere, hence a pressure difference is created between the sampling end 51 and ports 59. The pressure difference helps to direct the exhaled gases towards the detecting material at the attachment end 56.

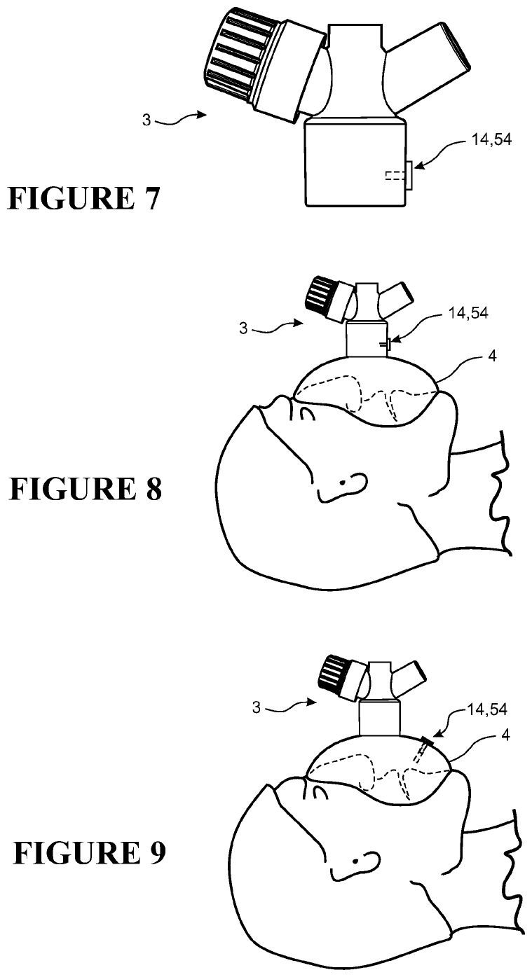

It should also be appreciated that the breath indicator 14 or 54 or 204, may be utilised directly with a face mask or other such patient interface, or may be positioned for receipt by a component of a breathing assistance apparatus, such as a T-piece, as shown by FIGS. 7, 8, 9. The breath indicators 14, 54, 204 of this invention provide for a removable indicator that may be received or attached to a part of a breathing assistance apparatus, with a system for placing the sensor close to the region of patient where gases are being exhaled, or alternatively where a sampling end or collector is provided for directing exhaled gas from a patient to a sensor for providing an indication of exhaled gas components (e.g. CO₂).

The breath indicator 14, 54, 204 as provided for herein can provide for an indication of an inhalation and/or exhalation state of a user, with the breath indicator 14, 54, 204 comprising a detecting material capable of changing colour from a first colour to a second colour according to inhalation and exhalation. As shown in the configurations of FIGS. 4A, 4B, 6A, 6B the detecting material may be provided upon the body with at least one indicium provided as an array or arrangement or sequenced stepping so as to enhance or emphasise a change in colour of the detecting material in response to detection of a user's breathing gases (such as carbon dioxide). However, in an alternative form, such as that illustrated by FIGS. 10A, 10B, 11-13, 14-18, the detecting material can be housed within a body of the breath indicator 14, 54, 204. The body 50 comprises a first port 52 configured for receipt of a user's inhalation and exhalation gases and at least a second port 59 to provide for a venting of said gases from said body 50. A component, such as that indicated as item 104, comprising of at least one indicia or one background to an indicium changeable as to contrast according to a change in colour of said detecting material with said user's inhalation and exhalation gases can also be provided.

As noted with respect to earlier configurations, the detecting material is advantageously a carbon dioxide detecting material or detector. The detecting material may have a first colour when a carbon dioxide concentration is about the same or substantially similar to atmospheric air, or when the detecting material is exposed to a gas containing or comprising up to about 0.03% or about 0.04% carbon dioxide (atmospheric). The detecting material may be of a second or a different colour when carbon dioxide concentration is greater than atmospheric air, or when the detecting material is exposed to gas containing about 2.5% or greater of a carbon dioxide concentration. Optionally, a first colour may be one of: blue or purple. Optionally, a second colour may be one of: yellow or orange.

A change from a first colour or an initial colour or an initial visual state to a second colour or a subsequent colour or a subsequent visual state may be based on a chemical reaction of said detecting material. Advantageously, the chemical reaction may be reversible.

The change from a first colour or an initial colour or an initial visual state to a second colour or a subsequent colour or a subsequent visual state should be a visually observable change.

In particular, the indicia can be provided for a greater contrast of colour or greater visibility or detection of a change in colour of the colour changeable detecting material. The change in colour of the detecting material is used to indicate either of a detected greater or lesser concentration of carbon dioxide relative to atmospheric carbon dioxide concentration.

Accordingly, the indicia can provide for a first contrast state or first visual state with respect to said detecting material when carbon dioxide concentration is the same or less than an atmospheric concentration of carbon dioxide, and/or a second contrast state or subsequent visual state with respect to said detecting material when carbon dioxide concentration is the greater than an atmospheric concentration of carbon dioxide. The first and second indicia contrast states or visual states may provide for degrees of visibility or predetermined shades of difference between such first and second states.

Advantageously, a second indicia contrast state or visual state may provide for a greater visibility or observability (i.e. appears more easily visible or optically detectable) with respect to the detecting material. Or, the second indicia contrast state or visual state may be significantly more visible than first indicia contrast state or visual state. The first indicia contrast state or visual state may be when said detecting material is a first colour or an initial colour or initial visual state.

The first indicia contrast state or initial visual state may correspond with when the detecting material is exposed to gas containing or comprising up to about 0.03% or 0.04% carbon dioxide (atmospheric), or when a carbon dioxide concentration is substantially the same as atmospheric air.

As noted previously, a change from a first indicia contrast state or initial visual state to a second indicia contrast state or subsequent visual state can be based on a chemical reaction of the detecting material.

In-use, a user or observer of the change in contrast state or colour states should be able to detect or observe a change from a first indicia contrast state or initial contrast state to a second indicia contrast state visually or subsequent visual state or optically (e.g. the colour change or contrast states are enhanced and are optically observable by a person).

At least one indicia can be a graphical representation of a pair of lungs, wind, a heart or a graphical smiley face, as shown in FIG. 19. It will be appreciated that other indicia may be used as appropriate.

In some configurations, the indicia may be printed or mounted directly upon said component. For example, an indicia can be printed or mounted directly upon a detecting material. Optionally, the detector material may visually surround said indicia, or alternatively the indicia may be located or positioned substantially wholly within the perimeter of a detecting material component. Alternatively, the detector material may visually border more than one edge or perimeter portion of said indicia. In this manner, it is intended to provide for a visually clearer contrast state or visually detectable change in colour of the detecting material with respect to the indicia, and thereby provide for an improved ease of detection by an observer of the breath indicator detecting material state or condition.

The component may be one or more of: the detecting material, a breath indicator cap C, 61 or another separate component of a breath indicator assembly.

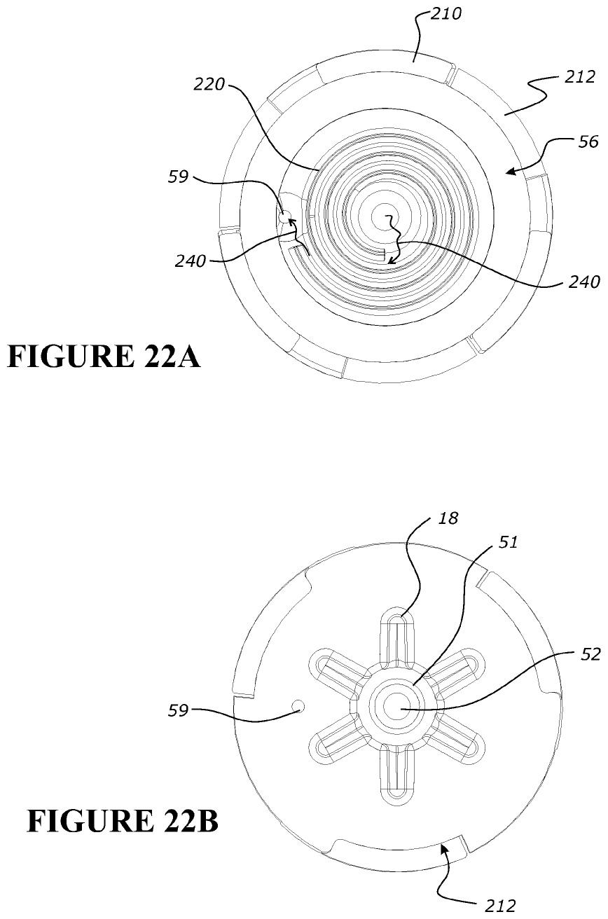

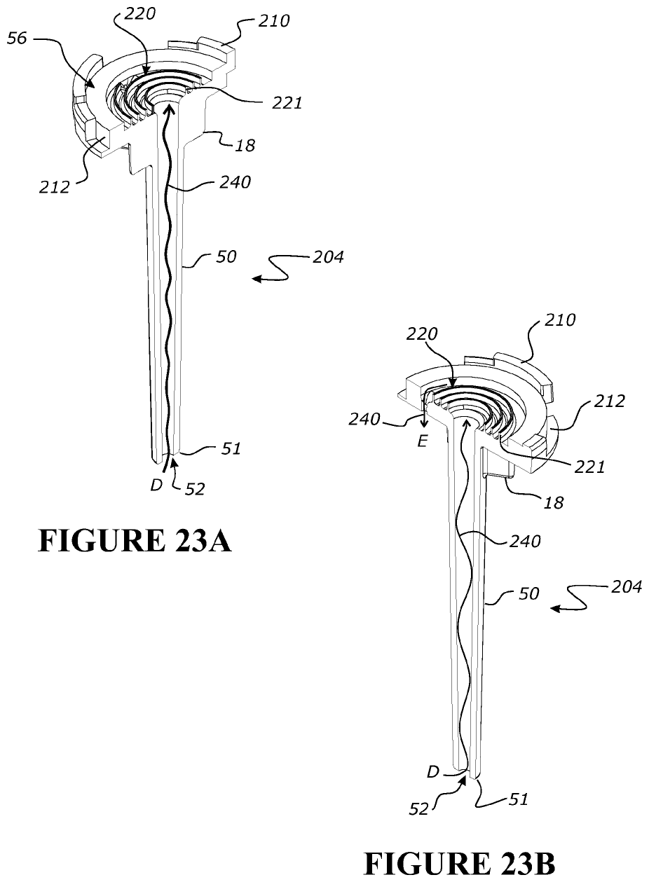

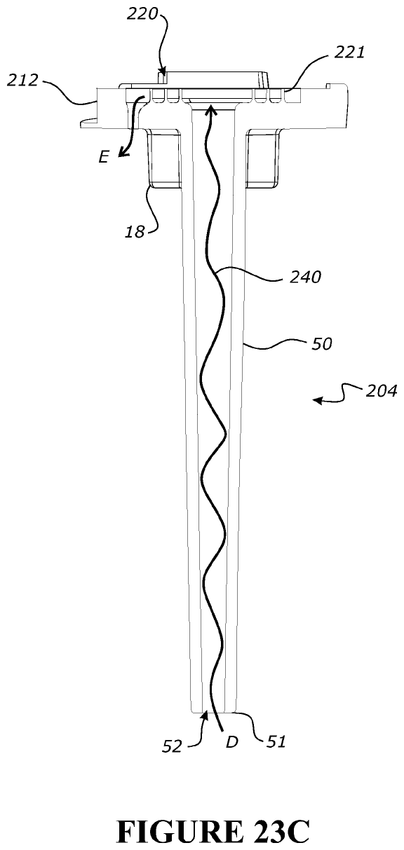

As is for example shown by FIGS. 10A, 10B, 11-13, 14-16, 17-18, 20-25, the breath indicator 54, 204 can further comprise of: an elongate body 50, a cap C, 61 and a sensor 55 comprising the detecting material. The sensor is located to be exposed to a gas flow through the elongate body 50 into a region housed by the cap. See for example the generalised gas flow path indicated by D, 240 and E, 240 in

FIGS. 12 and 15, and 23C illustrating a gas flow into the elongate body 50 of the breath indicator 54, 204, the gas flowing up through the elongate body 50, and venting to atmosphere out through the one or more ports 59.

In various configurations, the breath indicator 54, 204 can be provided with a cap C, 61 which can include or comprise of a radially inwardly extending projection portion 100 for holding or retaining the cap C, 61 and the elongate body 50 together in an engaged configuration.

The projection portion 100 may engage with an exterior portion of said elongate body 50. For example, the projection portion 100 may be configured to extend in a circumferentially inward manner around the cap C, 61. In some configurations, the cap C, 61 is receivable of an end 101 of the elongate body 50, with the cap C, 61 configured to be retained upon the end of said elongate body 50.

The end 101 of the elongate body 50 which may receive the cap C, 61 may also be an end of the elongate body 50 which is configured to receive or locate the sensor.

The end 101 of said elongate body 50 may be configured to provide a platform region for receiving or locating of the sensor.

With the cap C, 61 engaged with the end 101 of the elongate body 50, there can be provided a housing region 102 for retaining the sensor therein.

The elongate body 50 comprises a hollow passageway extending between ends of the elongate body 50, with a first end of said elongate body 50 provided as a gas sampling end 51 and a second end of said elongate body 50 provided as a region for receipt or locating of the sensor.

The second end can include the platform region upon which the sensor is to be received or located, with the hollow passageway terminating into that platform region, to thereby deliver a flow of sampled gas to the sensor for detection.

The detecting material can be received or located in a void space 102 provided between an end 101 of said elongate body 50 and an internal wall surface 103 of the cap C, 61 (when the cap is in an in-situ or in an engaged or an assembled configuration with the elongate body).

A component, such as that indicated by item 104, with at least one indicia may be received or located in the void space 102.

The component can be one or more of: the sensor, detecting material, cap, or the component can be provided as a separate item from each.

The cap is advantageously formed or manufactured so as to be, at least in part, of an optically transparent material. The cap, which may be a shell or a lid portion of the cap C, 61) may include optical magnification properties or capabilities for easier viewing of the indicia or component or detecting material (or all of these) when in their housed position.

The elongate body 50 may comprise an open end or a gas inlet 52 and a flow path or passageway or conduit extending from said open end 51 or the gas inlet to the sensor which is located in the void space 102.

The open end 52 or gas inlet is provided as a gas sampling or gas receiving end of the elongate body 50.

The flow path or passageway or conduit provides for a fluid (or gas) communication pathway between the gas sampling end 51 or the gas sensing end (or the sensor end) of the elongate body 50.

In some configurations, the elongate body 50 has a terminal end 105 of the flow path or passageway or conduit extending from the open end 52. The terminal end 105 is terminated by the cap C, 61 or a lid or other closed portion (e.g. when the cap C, 61 is in-situ or in an engaged or an assembled configuration with said elongate body). The terminal end 105 advantageously is provided with at least one gas flow port 59 to atmosphere for discharging a gas flow directed from a gas sampling end of said elongate body 50 through said terminal end. The gas flow port 59 is clearly illustrated in FIG. 15, as well as in FIG. 10B, including 20A, 23C, 24D. Such a port 59 or ports 59 are present in the other configurations, such as those shown in FIG. 10A, 11-13, 14.

The one or more gas flow port(s) 59 are provided as apertures. The one or more gas flow ports 59 may control the rate of gas flow or venting from said breath indicator 14, 54, 204.

The one or more gas flow ports 59 may be of a pre-determined size so as to provide a controlled or a pre-determined flow rate of gas which can discharge or vent from the breath indicator 14, 54, 204. Advantageously, gas flow rate may be controlled or limited to, for example, about 1 litre/minute. The port 59 may be, for example, a circular aperture of a diameter of about 0.7 mm. The port may have a diameter of about 0 mm to about 1 mm.

The one or more gas flow ports 59 may be substantially circular shaped aperture(s). In some configurations, there may be a single (one or sole) gas flow port 59 provided.

The one or more gas flow ports 59 are provided through a portion of the elongate body 50 that is in fluid (or gas) communication with a gas flow to be sampled or sensed. In this manner, with ports 59 provided downstream of the gas sampling end 51 and/or downstream of the sensor, the sampled and sensed gas can be vented to atmosphere.

One or more of the gas flow ports 59 may be located about or adjacent a stopping flange 58 portion of the elongate body 50.

The stopping flange 58, may be an enlarged region or portion of the elongate body 50, sized and/or shaped to provide a seat or physical stop to over-insertion of the breath indicator 14, 54, 204 into a device.

The stopping flange 58 can be a region or portion of the elongate body 50 that is engageable with a portion of a device to which the breath indicator 14, 54, 204 is to be attachable or engageable. In this manner, the stopping flange 58 provides a locatable position of the elongate body 50 with respect to, or relative to, the device. Furthermore, such a stopping flange 58 provides for a maximum or a predetermined length (or depth) of insertion of the elongate body 50 into such devices. The stopping flange 58 may be utilized with a tapered elongate body 50 to provide a smooth and aesthetically pleasing transition for the portion of the device housing the breath indicator 14, 54 to the gas sampling portion, which is more easily insertable to the required depth.

In one example configuration, the elongate body 50 may be about 40 mm in length, with the cap being a further 5 mm in height or length.

In further configurations, the breath indicator 54, 204 may further comprise a body comprising a gas sampling portion or end 51 and a gas sensing portion or end, with the gas sampling portion or end 51 being in fluid communication with the gas sensing portion. The gas detector may be housed within the gas sensing portion or detector portion 56, with the gas detector comprising a material providing for a visually detectable change in colour in response to a concentration of a gas to be detected, and wherein a pre-determined indicia is provided directly upon said gas detector.

In the configuration as shown in FIGS. 11 to 19, the breath indicator 54, 204 comprises an elongate body 50 having a gas sampling portion end 51 and a gas sensing portion end or detector portion 56, with the gas sampling portion end 51 being provided in fluid communication with the gas sensing portion end or detector portion 56. The gas detecting material is to be housed within the gas sensing portion end or detector portion 56, with the gas detecting material 230 providing for a visually detectable change in colour in response to a concentration of a gas to be detected, and wherein a pre-determined indicia is provided directly upon said gas detector.

The pre-determined indicia 104 may be of a pre-determined shape, or a colour or a shape and colour, or visual state, as shown in FIG. 19.

The indicia 104 may be of a shape resembling an icon. The indicia or icon may be indicative of breathing, or lungs, or wind, or a heart or a graphical smiley face or other positive display of a detected carbon dioxide concentration or another visual cue. For example, see those as illustrated in FIG. 19.

The indicia 104, or one or more indicia 104, may be directly imprinted or printed or laid upon a gas detector or a gas detector material, or can be provided as a separate component which is to be directly located or laid upon said gas detector when provided as a separate component from the gas detector. In this manner, a direct overlay or a direct printing of the indicia on the detecting material is provided for, and in doing so a change in the colour of the detecting material may be able to be more readily observable or detectable due to a contrast in the colour of the detecting material and the indicia.

As noted previously, the gas detecting material acts as a sensor, with the sensor being located within a gas flow or exposed to a gas flow flowing through or about the body, thereby bringing the sensor into contact with the gas to be detected or sensed.

The body 50 of the indicator 54, 204 can be of an elongate form body. The elongate body can taper from a relatively smaller outer diameter substantially at or toward said gas sampling portion end 51 to a relatively larger outer diameter substantially at or toward said gas sensing portion end. In some configurations, the gas sampling portion end 51 may comprise a lead-in portion 106.

The lead-in portion 106 can be a region of the elongate body 50 which is of a substantially narrowed (or more narrowed) outer diameter, or such a lead-in portion 106 can be a tip or an end of the elongate body 50 that comprises of a chamfered edge or an edge with substantially radially inward curvature so as to assist with access or initial insertion of the gas sampling end 51 into a device, such through as a duck-bill valve of a device.

A substantially tapered configuration of the body (such as an elongate body of said indicator) can extend from at or substantially toward the gas sampling portion end 51 toward the gas sensing portion or detector portion 56, about one quarter, or about one third, or about one half, two-thirds, three-quarters, or a whole of, the length of the elongate body 50.

The cap C, 61 which can be provided as a housing to the gas detecting material can be a removable or detachable part from the indicator 54, 204. The indicia may be printed or otherwise applied to the cap.

The indicia when provided as a separate component can also be a separately removable part so as to be replaceable (or serviceable) or alternatively, where the indicia is provided directly upon (e.g. printed or marked directly on the gas detecting material), the detector material (with indicia thereon) can be removed and replaced from the breath indicator if needed. Such a configuration may allow for reuse of the breath indicator.

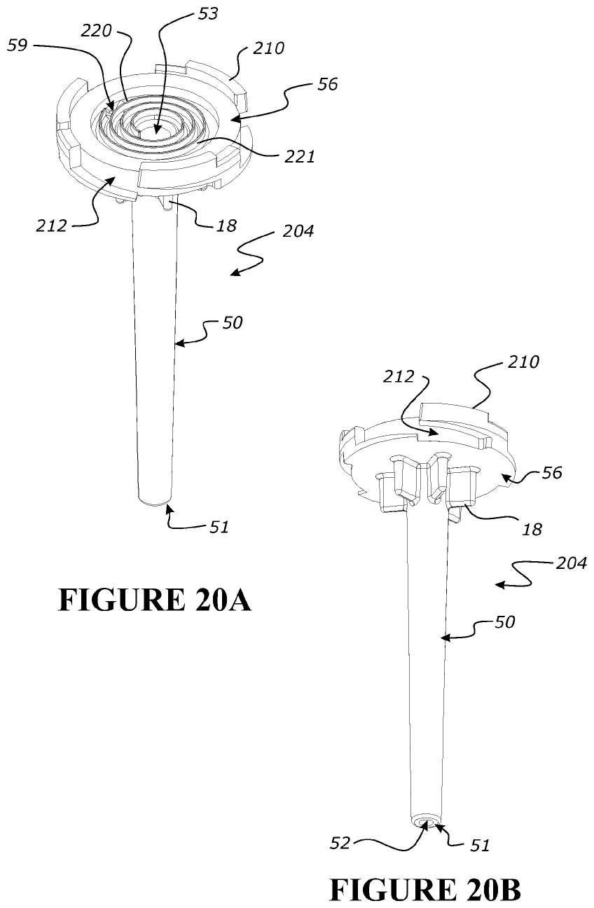

In a further configuration as shown in FIGS. 20 to 25, the breath indicator 54, 204 is provided with a single or sole gas flow port 59 for venting a gas from said indicator to atmosphere. In this manner, a controlled gas flow rate for venting or discharge of gas from the indicator may be provided.

In a further configuration, the breath indicator 54, 204 provided for a body comprising a gas sampling portion end 51 and a gas sensing portion end, where the gas sampling portion end 51 is provided in fluid communication with the gas sensing portion end or detector portion 56, with a gas detector housed within the gas sensing portion end or detector portion 56. The gas detector comprising a detector material providing for a visually detectable change in colour in response to a concentration of a gas to be detected, and wherein a pre-determined indicia is provided directly upon said gas detector. Such indicia may be as described herein.

In a further configuration, a breath indicator 54, 204 is provided for having a body comprising a gas sampling portion end 51 and a gas sensing portion end or detector portion 56, with the gas sampling portion end 51 provided in fluid communication with the gas sensing portion or detector portion 56 via an elongate body 50. The gas sensing portion end or detector portion housing a gas detector or a sensor, with the gas detector being of a material providing for a visually detectable change in colour or visual state in response to a gas parameter, such as a concentration, of a gas to be detected. The gas sensing portion end or detector portion 56 is positioned or provided distal from the gas sampling portion end. The elongate body 50 is a member being of a substantially tapered configuration, tapered from a relatively smaller outer diameter at or substantially toward said gas sampling end 51 to a relatively larger outer diameter as the elongate body progresses toward the gas sensing portion end or detector portion 56.

In a further configuration, there is provided a breath indicator 14, 54, 204 as shown in FIGS. 1 to 9, 10 to 19, and 20 to 25, respectively, for providing an indication of an inhalation and/or exhalation state of a user. The breath indicator 14, 54, 204 comprises a body 50 comprising a gas sampling portion 51 configured for receipt of a user's inhalation and/or exhalation gases, and an indicator portion 16, 56, the gas sampling portion 51 in fluid communication with said indicator portion 16, 56. The indicator portion 16, 56 comprising at least a first region 15, 205 and at least a second region, such as that indicated as item 104, the first region comprising a gas parameter detecting material 230 capable of changing between an initial visual state and a subsequent visual state indicative of detection of a gas parameter of inhalation and/or exhalation gases of said user; and wherein the initial visual state and/or the subsequent visual state of the first region 15, 205 visually contrasts with a visual state of at least the second region, such as that indicated as item 104.

The first region 15, 205 may be shaped in the form of an indicium. Alternatively, or in addition, the second region (such as that indicated as item 104) may also be shaped in the form of an indicium. Either the first region and/or the second region may substantially surround the respective second region and/or the first region.

The second region may comprise a static or substantially unchanging visual state. Or, the second region may comprise a gas parameter detecting material capable of changing from an initial visual state to a subsequent visual state, the change indicative of a detected gas parameter of inhalation and/or exhalation gases of a user.

The indicator portion 15, 56 may comprises at least one further region, the further region may comprise a static or substantially unchanging visual state. Or, the further region may comprise a gas parameter detecting material capable of changing from an initial visual state to a subsequent visual state, the change indicative of a detected gas parameter of inhalation and/or exhalation gases of a user. The further region may be in the form of an indicium.

One or more of the first region 15, 205, and/or the second region (such as that indicated as item 104), and/or a further region of the indicator portion 16,56 may be substantially adjacent with each other, or are substantially surrounding of one or more of the first region and/or the second region and/or a further region.

At least one of the first region 15, 205 or the second region, or a further region, may be provided as a background visual state. The background visual state may be provided by a gas parameter detecting material capable of changing from changing from an initial visual state to a subsequent visual state, being indicative of a detected gas parameter of inhalation and/or exhalation gases of said user. Or, the background visual state may be a static or substantially unchanging visual state.

The indicium as described above may be of a pre-determined shape and/or colour and/or contrast and/or visual state. The indicium may resemble an icon. The indicium may be indicative of breathing, lungs, wind, a heart, a graphical smiley face or other positive display of a detected gas parameter or another visual cue.

The body 50 may be an elongate body, and may taper from a relatively smaller outer or external diameter substantially at or toward the gas sampling portion 51 to a relatively larger outer or external diameter substantially at or toward the indicator portion 16, 56. The gas sampling portion 51 may comprise a lead-in portion, being at least one of (i) a region of said elongate body having a comparatively narrowed external diameter, or (ii) a tip or end of said elongate body comprising a chamfered edge or substantially radially inward curvature so as to assist with access of said gas sampling end into a device.

A substantially tapered configuration of the body 50 extending from the gas sampling portion 51 toward said indicator portion 16, 56 may be about one quarter, or about one third, or about one half, two-thirds, three-quarters, or a whole of, the length of the elongate body 50.

The indicator portion 16, 56 may comprise a cap C, 61 configured to substantially cover the gas parameter detecting material 230 and complete a gas pathway 240 between the gas sampling portion 51 and the indicator portion 16, 56.

The indicium may be imprinted, printed or laid upon the cap C, 61, or may be provided as a separate component which is locatable on said cap when provided as a separate component.

The cap C, 61 may be removable or detachable from the body 50, and may be rotatably engageable with the body, and/or one or more of: engaged to the body via one or more locking cam(s), threadably attachable to the body, push fit to the body.

The cap C, 61 and body 50 may be configured such that the engagement is by helical rotation. The cap C, 61 and the indicator portion 16, 56 may comprise one or more complementary radial ramp structures configured to provide the helical rotation.

The indicator portion 16, 56 may comprise at least one tab 210 configured to engage the cap C, 61.

The cap C, 61 may comprise a slot or aperture 212 configured to accept the at least one tab 210 of the indicator portion 16, 56.

The tab 210 may provide for a standoff feature configured to locate the cap C, 61 in a predetermined position. The predetermined position may be a height relative to the gas parameter detecting material 230. Alternatively, or in addition, the predetermined position may be a rotated location or angular position relative to the indicator portion 16, 56.

The cap C, 61 may be configured to substantially enclose the indicator portion 16, 56.

The cap C, 61 may be substantially transparent or translucent to provide for a visual identification of a changing colour and/or visual state of the gas parameter detecting material 230.

The indicator portion 16, 56 may comprise at least one port configured to vent to atmosphere. Alternatively, or in addition the cap C, 16 may comprise at least one port configured to vent to atmosphere.

The body may comprise a gas pathway 240 between the gas sampling portion 51 and the indicator portion 16, 56, the gas pathway 240 configured to expose a volume of gas to the gas parameter detecting material 230.

The gas pathway 240 may be configured to uniformly direct exposure of a volume of a user's inhalation and/or exhalation gases with said gas parameter detecting material 230.

The gas pathway 240 may be configured to increase a gas parameter detecting material 230 surface area in contact with a user's inhalation and/or exhalation gases.

The gas pathway 240 may comprise a channel 220, and the cap C, 61 may be configured to complete the gas pathway 240 between the gas sampling portion 51 and the indicator portion 16,56 when attached to said body 50.

The gas pathway 240 may be elongate, and may be an elongate channel 220 or passageway. The pathway may be spiralled, for example spiralled radially outwardly from an outlet of the gas sampling portion 51, or an inlet into the indicator portion 16, 56.

The gas pathway 240 may provide for a gas path which is of a substantially greater distance than an internal radius of the indicator portion 16, 56.

The gas pathway 240 may provide for an indirect gas path, being of a substantially greater distance than a direct or straight-line distance between at least one port 59 configured to vent to atmosphere from the indicator portion 16, 56 and an inlet to said indicator portion from said gas sampling portion 51.

The gas pathway 240 may extend between at least one port 59 configured to vent to atmosphere from the indicator portion 16, 56 and an inlet to the indicator portion from the gas sampling portion 51.

The gas pathway 240 may provide for a substantially continuous path between an outlet of the gas sampling portion 51 or an inlet to the indicator portion 16, 56, and at least one port 59 configured to vent to atmosphere from the indicator portion 16, 56.

The at least one port 59 configured to vent to atmosphere from the indicator portion 16, 56 may be located adjacent a stopping flange portion 18 of the body 50.

The stopping flange 18 may be an enlarged region or portion of the body 50.

The stopping flange 18 may be a region or portion of the body 50 that is engageable with a portion of a device to which the breath indicator is attachable or engageable, to provide for a located position of the body 50 relative to a device or to provide for a maximum or a predetermined length (or depth) of insertion of the elongate body 50 into a device.

The gas parameter detecting material 230 may be one or more of: a gas detecting material, a gas composition detecting material, a gas humidity detecting material, a gas temperature detecting material, a carbon dioxide concentration detecting material or detector.

The gas parameter detecting material 230 may be of a first colour and/or an initial visual state when a gas parameter is about the same or substantially similar to atmospheric air. The gas parameter detecting material 230 may be of a first colour and/or an initial visual state when exposed to gas containing or comprising about 0.03% to about 0.04% carbon dioxide (atmospheric). The gas parameter detecting material 230 may be of a second colour and/or of a subsequent visual state when a gas parameter is greater than atmospheric air. The gas parameter detecting material 230 may be of a second colour and/or of a subsequent visual state when exposed to gas containing about 2% to about 3% or greater of a carbon dioxide concentration.

The gas parameter detecting material 230 may be of a second colour and/or of a subsequent visual state when exposed to gas containing greater than about 0.04% of a carbon dioxide concentration, or greater than about 0.2% of a carbon dioxide concentration, or greater than about 2.5% of a carbon dioxide concentration. The gas parameter detecting material 230 may be of a second colour and/or of a subsequent visual state when exposed to gas containing greater a carbon dioxide concentration than atmospheric air.

A first colour and/or of an initial visual state of the gas parameter detecting material 230 may be one of: blue or purple. A second colour and/or a subsequent visual state of the gas parameter detecting material 230 may be one of: yellow or orange.

A change from a first colour and/or an initial visual state to a second colour and/or a subsequent visual state of the gas parameter detecting material 230 may be based on a chemical reaction of the gas parameter detecting material 230. The chemical reaction may be reversible.

A change from a first colour and/or initial visual state to a second colour and/or subsequent visual state of the gas parameter detecting material 230 may be visually observable. The indicia as described herein may provide for a greater contrast of colour or greater visibility with respect to the gas parameter detecting material 230 to indicate either of a detected greater or lesser gas parameter relative to atmospheric air.

The indicia may provide for: i. a first colour and/or an initial visual state with respect to the gas parameter detecting material 230 when a gas parameter is the same or less than an atmospheric gas parameter, and/or ii. a second colour and/or a subsequent visual state with respect to the gas parameter detecting material 230 when a gas parameter is the greater than an atmospheric gas parameter.

An initial and a subsequent indicium colour and/or visual states may be degrees of visibility. A subsequent indicium colour and/or visual state may be of greater visibility or observability with respect to the gas parameter detecting material 230. A subsequent indicium colour and/or visual state may be significantly more visible than an initial indicium colour and/or visual state. There may be an initial indicium colour and/or initial visual state when the detecting material 230 is a first colour and/or a first visual state. There may be an initial indicium colour and/or an initial visual state when the gas parameter detecting material 230 is exposed to gas containing or comprising about 0.04% or less carbon dioxide (atmospheric). There may be an initial indicium colour and/or an initial visual state when carbon dioxide concentration is substantially the same or less than atmospheric air. A change from an initial indicium colour and/or an initial visual state to a subsequent indicium colour and/or a subsequent visual state may be based on a chemical reaction of the gas parameter detecting material 230. A change from an initial indicium colour and/or an initial visual state to a subsequent indicium colour and/or subsequent visual state may be a visually or an optically observable change in contrast.

FIGS. 20 to 25 illustrate a further embodiment of the breath indicator 204. In this embodiment, the breath indicator 204 may facilitate a directed gas flow path.

Advantageously, such a directed gas flow path may provide for optimised flow of gas in contact with a gas parameter detecting material 230. A directed gas flow path may allow gas to more uniformly contact or be more uniformly distributed or be more completely or substantially evenly distributed for contact with a gas parameter detector, which may facilitate improved detection of inhalation and exhalation gases.

FIGS. 20A to 23C illustrate upper and lower perspective views of this embodiment of the breath indicator 204. Elongate body 50 may comprise an elongate cylinder as described herein, with a sampling end 51 comprising a gas inlet 52 and a stopping flange 18.

The stopping flange 18 may be an enlarged region or portion of the elongate body 50, sized and/or shaped to provide a seat or physical stop to over-insertion of the breath indicator 204 into a device, such as into a duck-billed valve of a patient interface.

In an example embodiment, the stopping flange 18 may comprise ridges or walls extending radially outwardly from the elongate body 50. The stopping flange 18 may alternatively or in addition comprise a radially outwardly extending section substantially perpendicular to an axial direction of the elongate body 50.

The breath indicator 204 may comprise an attachment end 56 located distal from the sampling end 51. The attachment end 56 may comprise one or more features configured to direct a flow path 240 of gas in the attachment end 56.

In an example embodiment, the detection end may include a channel 220 extending from an open end of the passageway 53 of elongate body 50 distal to a gas inlet 52 at sampling end 51.

The channel 220 may terminate at a gas port 59. In the example embodiment shown, the channel 220 is a substantially spiral configuration, extending from gas passageway 53 to gas port 59 in a direction substantially perpendicular to a direction of the gas passageway.

It will be understood that other suitable arrangements for channel 220 are envisaged. For example, a substantially zig-zag or curved or elongate pathway, to provide an optimal flow of gas to contact the detection material 230 and to cause a colour change and/or contrast. The channel 220 may extend a gas path 240 from gas passageway 53 to gas port 59.

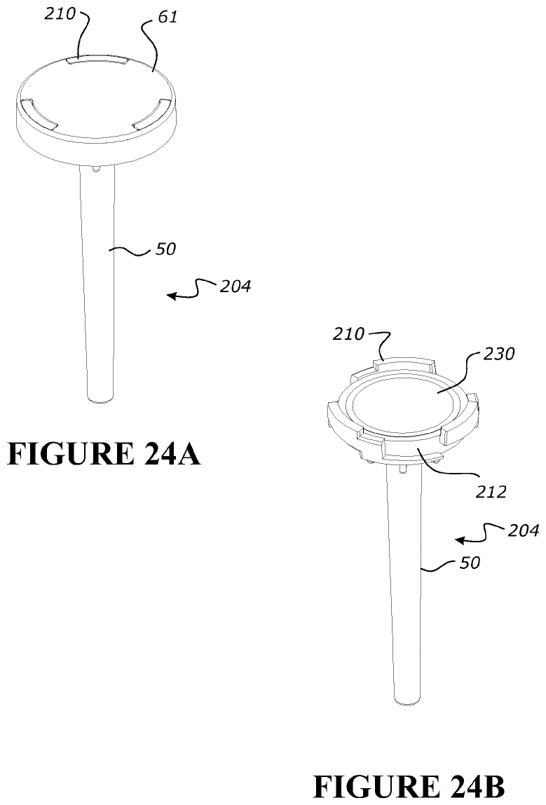

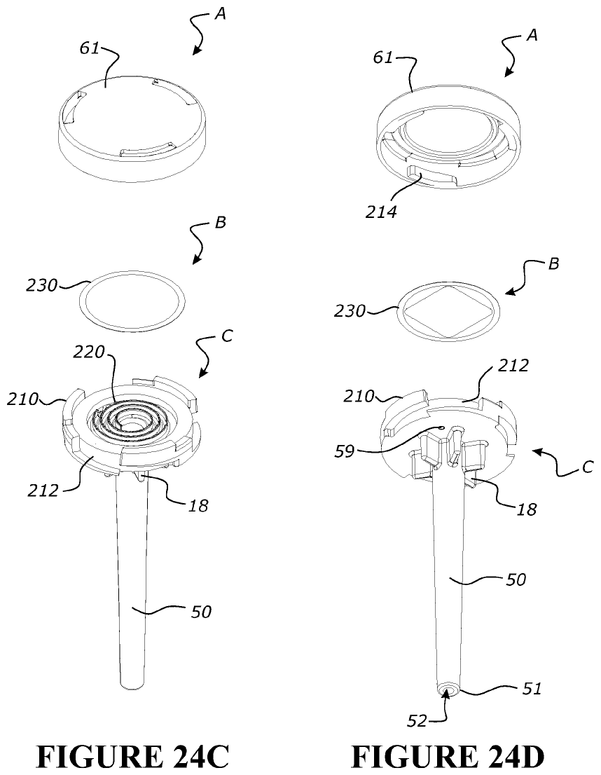

FIGS. 24A to 24D illustrate an embodiment of the breath indicator 204 with a cap 61 and detector material 230. It will be understood that any of the indicia arrangements described herein, for example in regard to FIG. 19, may be included on or in detector material 230. FIG. 24A illustrates the breath indicator 204 with a cap 61 positioned in place on elongate body 50. FIG. 24 B illustrates the breath indicator 204 with a cap removed, showing detector material 230 in place.

FIGS. 24C and D is an exploded perspective diagram showing cap 61 at A, detector material 230 at B, and body 50 at C.

In an example embodiment, cap 61 may be configured to substantially hold detector material 230 in place against channel 220 described above. The cap 61 may be configured to aid in sealing detector material 230 over channel 220.

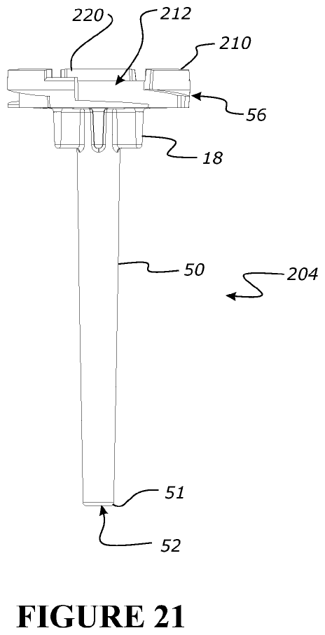

In an example embodiment the detector material 230 may be stretched over the channel 220, which may be in the form of a spiral shape as illustrated in FIGS. 20 to 25. The walls or structures 221 defining the channel 220 may protrude or substantially resemble a domed arrangement if viewed from the side as shown in FIG. 21. The detector material 230 may be stretched over the spiral channel 220, over the top of the walls/structures 221 forming the domed arrangement and towards the outer edges of attachment end 56. In this way, the detecting material 230 may be sealed in place, such that the only gas path 240 is via elongate body 50 from gas inlet 52, through channel 220, and out of port 59. Whilst the gas is traversing this gas path 240, the gas comes into contact with detecting material 230 stretched over channel 220, providing uniform interaction between the gas and the detecting material. In an alternative embodiment, the indicator 56 may not have an outlet or port 59, and the gas may diffuse or otherwise travel via the gas path 240 to the detecting material 230.

In an example embodiment the port 59 may be sized and/or shaped to create resistance of flow through the breath indicator. Alternatively, or in addition, the channel 220 as described herein may be sized and/or shaped to create a resistance to flow. It will be understood that any configuration that achieves resistance of flow may be utilised, i.e. a ratio of driving pressure to the rate of air flow whilst still achieving a therapeutic effect. The port 59 may also provide for easier exhalation of a patient when the breath indicator is in use with a patient interface.

Attachment end 56 may comprise a locking cam 212 or thread features configured to rotatably engage with cap 61 to provide for a secure connection and sealed configuration, where the detector material is encapsulated or sealed. Locking cams 212 may comprise a radial ramp or taper around attachment end 56 such that, when cap 61 is engaged with locking cam 212, cap 61 is pulled substantially toward gas passageway 53 and against detector material 230 disposed on channel 220. Cap 61 may alternatively or in addition comprise one or more tabs configured to engage with locking cam 212 as described herein.

The attachment end 56 may comprise locking features 210, which may be configured to protrude through cap 61 when in full engagement with locking cam 212 to provide a locking mechanism to secure cap 61 to elongate body 50. In the example shown in the Figures, three locking features 210 and three locking cams 212 are provided, however it will be understood that any number of locking cams 212 and/or locking features 210 could be provided to enable a securement and sealing of cap 61 to elongate body 50 of the breath indicator 204.

In the embodiment shown in FIGS. 20-25, a single port 59 is shown to provide a gas exhaust or vent point and positioned on a side of attachment end 56 opposite to the side comprising channel 220. Put another way, the port 59 is positioned as part of attachment end 56 and at an underside of said attachment end 56, opposite to that configured to receive cap 61.

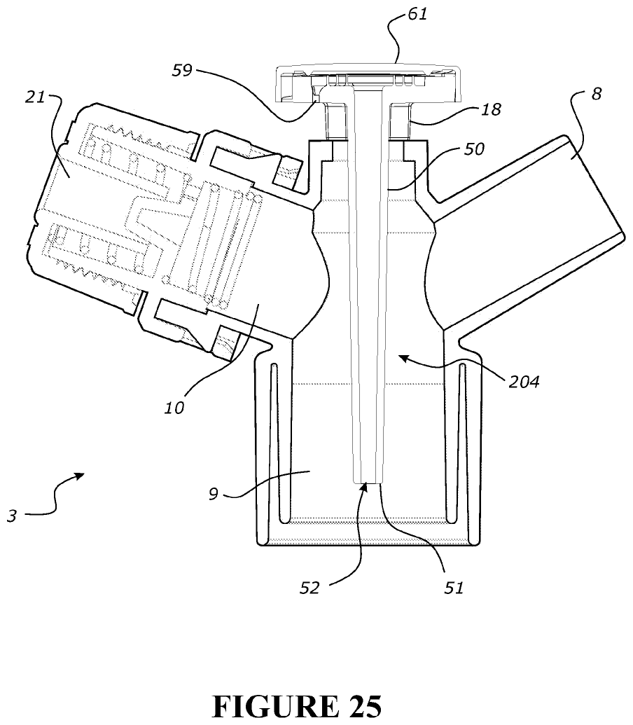

In an example shown in FIG. 25, the stopping flange 18 is provided to prevent the breath indicator 204 being inserted too far into a T-piece. Additionally, stopping flange 18 is configured such that it prevents the port 59 from being obstructed or occluded when the breath indicator 204 is inserted into the T-piece.

Whilst the example embodiment shown in FIGS. 20 to 25 comprises a single port 59, it will be understood that any number of ports 59 could be provided to provide for a suitable vent rate in the elongate body or in the cap 61.

In an alternative embodiment, no outlet may be provided, i.e. a venting rate lower bound may be substantially zero. Even if there is no outlet (i.e. no port 59), some inhalation and/or exhalation gases will be detected by detecting material 230. Put another way, the breath indicator will function without a controlled vent or port 59.

In an example embodiment, an upper threshold for gas venting via port 59 depends on the value of gas venting at which the T-piece as described herein loses operability and therapeutic effect. In other words, the vent rate via port 59 needs to be within the operational parameters of PEEP and PIP. Within typical operating parameters, assuming flow set from about 8-10 L/min, upper vent threshold may be about 2.5 to about 3 L/min. This range may be suitable for neonatal use, however an increased flow rate and vent rate may be provisioned for a T-piece configured for use with an adult.

As outlined above, channel 220 may be provided as a substantially spiralled structure. A substantially spiralled wall 221 may form vertical sides of said channel 220. A base of the channel 220 may be formed by providing said walls onto or as part of the attachment end 56 of elongate body 50. Gas parameter detector material 230 may substantially seal to form a top or upper surface of the channel, forming a substantially sealed gas passageway configured to extend gas passageway 53 to port 59. Such a configuration may enable a spiral gas path 240 providing a more uniform contact, where gas is in contact with detector material 230 between gas passageway 53 and gas port 59.

The gas pathway may be an elongate (optionally spiral) pathway or channel 220, and may be configured for distribution of gas substantially across the detector material 230. This may facilitate more uniform or even contacting of the gas with the detector material 230. This may advantageously provide for a uniform and faster detecting material change with a change in gas parameter.

As described herein, an elongated pathway may lengthen the travel path of the gas between the detector portion inlet and the port (vent) 59. This may advantageously improve the contact of the gas with the detector material 230 before the gas reaches the vent 59, and may increase the residence time and/or the contact area of the gas provided in contact with the detector material 230.

As outlined above, cap 61 may aid in sealing detector material 230 to channel 220.

However, in an alternative embodiment, detector material 230 may seal to channel 220 via other means, such as a retention mechanism on attachment end 56. In an example embodiment, the detector material 230 may be stretched onto or over the channel 220 radially outwardly towards edges of the cap 61 and attachment end 56.

In an example embodiment, any of the T-pieces and/or breath indicators 14, 54, 204 as described herein may comprise one or more antibacterial surfaces. In an example embodiment, the surface of the breath indicator inside and outside the device is substantially antimicrobial or comprises antibacterial or anti-microbial properties. For example, the breath indicator 14, 54, 204 may be formed of a plastics material such as nylon, polycarbonate, polyethylene, polypropylene, etc. Additionally, or alternatively the breath indicator may be formed of a material comprising antimicrobial additives, coatings and/or patterned surfaces that provide antimicrobial properties.

The present invention may have particular application to an infant or neonate due to the ability to locate a sensor or sampling end for sampling of gases close to the point or region of exhalation by an infant or neonate. Infants and neonates have a particular problem in that their volume of exhaled breath is comparatively small compared to an adult, accordingly, if an indication of exhalation of a patient is to be made for an infant or neonate, it is useful that what breath is exhaled is sensed by a breath indicator.

Features from one or more of the above configurations and/or methods may be combined with features of one or more other configurations and/or methods. Additionally, more than one configuration and/or method may be used together during a process of respiratory support of a patient.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to.”

Where, in the foregoing description reference has been made to integers or components having known equivalents thereof, those integers are herein incorporated as if individually set forth.

The invention may also be said broadly to consist in the parts, elements and features referred to or indicated in the specification of the application, individually or collectively, in any or all combinations of two or more of said parts, elements or features.

Reference to any prior art in this specification is not, and should not be taken as, an acknowledgement or any form of suggestion that that prior art forms part of the common general knowledge in the field of endeavour in any country in the world.

Certain features, aspects and advantages of some configurations of the present disclosure have been described with reference to use of the gas humidification system with a respiratory therapy system. However, certain features, aspects and advantages of the use of the gas humidification system as described may be advantageously be used with other therapeutic or non-therapeutic systems requiring the humidification of gases. Certain features, aspects and advantages of the methods and apparatus of the present disclosure may be equally applied to usage with other systems.

Although the present disclosure has been described in terms of certain configurations, other configurations apparent to those of ordinary skill in the art also are within the scope of this disclosure. Thus, various changes and modifications may be made without departing from the spirit and scope of the disclosure. For instance, various components may be repositioned as desired. Moreover, not all of the features, aspects and advantages are necessarily required to practice the present disclosure. Accordingly, the scope of the present disclosure is intended to be defined only by the claims that follow.

1. A breath indicator for providing an indication of an inhalation and/or exhalation state of a user, said breath indicator comprising:
a body comprising a gas sampling portion configured for receipt of a user's inhalation and/or exhalation gases, and an indicator portion, said gas sampling portion in fluid communication with said indicator portion,
said indicator portion comprising at least a first region and at least a second region, the first region comprising a gas parameter detecting material capable of changing between an initial visual state and a subsequent visual state indicative of detection of a gas parameter of inhalation and/or exhalation gases of said user; and
wherein said initial visual state and/or said subsequent visual state of the first region visually contrasts with a visual state of at least said second region.

2. (canceled)

3. The breath indicator of claim 1, wherein the first region or the second region is shaped in the form of an indicium.

4. The breath indicator of claim 1, wherein either the first region and/or the second region substantially surrounds the respective second region and/or the first region.

5-9. (canceled)

10. The breath indicator of claim 1, wherein one or more of the first region and/or the second region and/or a further region of said indicator portion are substantially adjacent each other, or substantially surround one or more of the first region and/or the second region and/or a further region.

11-14. (canceled)

15. The breath indicator of claim 1, wherein at least one of said first region or said second region, or a further region, is provided as a background visual state, and wherein said background visual state is provided by a gas parameter detecting material capable of changing from an initial visual state to a subsequent visual state, being indicative of a detected gas parameter of inhalation and/or exhalation gases of said user.

16-20. (canceled)

21. The breath indicator of claim 1, wherein said body is an elongate body, and wherein said elongate body tapers from a relatively smaller outer or external diameter substantially at or toward said gas sampling portion to a relatively larger outer or external diameter substantially at or toward said indicator portion.

22. (canceled)

23. The breath indicator of claim 1, wherein said gas sampling portion comprises a lead-in portion, and wherein said lead-in portion is at least one of (i) a region of said elongate body having a comparatively narrowed external diameter, or (ii) a tip or end of said elongate body comprising a chamfered edge or substantially radially inward curvature so as to assist with access of said gas sampling end into a device.

24. (canceled)

25. The breath indicator of claim 1, wherein said indicator portion comprises a cap configured to substantially cover the gas parameter detecting material and complete a gas pathway between the gas sampling portion and the indicator portion, and said indicium is imprinted, printed or laid upon said cap, or is provided as a separate component which is locatable on said cap when provided as a separate component.

26-31. (canceled)

32. The breath indicator of claim 25, wherein the indicator portion comprises at least one tab configured to engage the cap, wherein the tab provides for a standoff feature configured to locate the cap in a predetermined position, and the predetermined position is a height relative to the gas parameter detecting material.

33-40. (canceled)

41. The breath indicator of claim 1, comprising a gas pathway configured to expose or contact or distribute a volume of a user's inhalation and/or exhalation gases with said gas parameter detecting material.

42. (canceled)

43. The breath indicator of claim 41, wherein the gas pathway is configured to increase a gas parameter detecting material surface area in contact with a user's inhalation and/or exhalation gases.

44. The breath indicator of claim 41, wherein the gas pathway comprises a channel, and the cap is configured to complete the gas pathway between the gas sampling portion and the indicator portion when attached to said body.

45-46. (canceled)

47. The breath indicator of claim 41, wherein said gas pathway is spiraled.

48. The breath indicator of claim 47, wherein said pathway spirals radially outwardly from an outlet of said gas sampling portion, or an inlet into said indicator portion.

49. The breath indicator of claim 41, wherein said pathway provides for a gas path which is of a substantially greater distance than an internal radius of said indicator portion.

50. The breath indicator of claim 41, wherein said gas pathway provides for an indirect gas path, being of a substantially greater distance than a direct or straight-line distance between at least one port configured to vent to atmosphere from said indicator portion and an inlet to said indicator portion from said gas sampling portion.

51-54. (canceled)

55. The breath indicator of claim 50, wherein the at least one port configured to vent to atmosphere from said indicator portion is located adjacent a stopping flange portion of the body, and wherein said stopping flange is a region or portion of said body that is engageable with a portion of a device to which said breath indicator is attachable or engageable, to provide for a located position of said body relative to a said device or to provide for a maximum or a predetermined length (or depth) of insertion of said elongate body into a device.

56-68. (canceled)

69. The breath indicator of claim 3, wherein said indicium provides for:
i. a first colour and/or an initial visual state with respect to said gas parameter detecting material when a gas parameter is the same or less than an atmospheric gas parameter, and/or
ii. a second colour and/or a subsequent visual state with respect to said gas parameter detecting material when a gas parameter is the greater than an atmospheric gas parameter.

70-77. (canceled)

78. A breath indicator for providing an indication of an inhalation and/or exhalation state of a user, said breath indicator comprising:
a body comprising a gas sampling portion configured for receipt of a user's inhalation and/or exhalation gases, and an indicator portion, said gas sampling portion in fluid communication with said indicator portion,
said indicator portion comprising a first region and a second region, one or both of the first and second regions comprising a gas parameter detecting material capable of changing between an initial visual state and a subsequent visual state indicative of detection of a gas parameter of inhalation and/or exhalation gases of said user;
wherein said initial visual state and/or said subsequent visual state of the first region visually contrasts with a visual state of at least said second region, and
wherein the body comprises a gas pathway between the gas sampling portion and the indicator portion, the gas pathway configured to expose a volume of gas to said detecting material.

79. A breath indicator for providing an indication of an inhalation and/or exhalation state of a user, said breath indicator comprising:
a body comprising a gas sampling portion configured for receipt of a user's inhalation and/or exhalation gases, and an indicator portion, said gas sampling portion in fluid communication with said indicator portion;
said indicator portion comprising at least a first region and at least a second region, the first region comprising a gas parameter detecting material capable of changing from an initial visual state to a subsequent visual state indicative detection of a gas parameter of inhalation and/or exhalation gases of said user; and
wherein said initial visual state and/or said subsequent visual state of the first region visually contrasts with a visual state of at least said second region, and
wherein said indicator portion comprises a cap configured to substantially cover the detecting material and complete a gas pathway between a gas sampling portion and the indicator portion.
